(19) United States
(12) Patent Application Publication
(10) Pub. No.: US 2013/0064901 A1
(43) Pub. Date: Mar. 14, 2013

(54) GENE EXPRESSION PROFILING FOR CLASSIFYING AND TREATING GASTRIC CANCER

(71) Applicants: Patrick Tan, Singapore (SG); Iain Tan, Singapore (SG)

(72) Inventors: Patrick Tan, Singapore (SG); Iain Tan, Singapore (SG)

(73) Assignee: Agency for Science, Technology and Research, Connexis (SG)

(21) Appl. No.: 13/450,423

(22) Filed: Apr. 18, 2012

Related U.S. Application Data
(60) Provisional application No. 61/476,698, filed on Apr. 18, 2011.

(51) Int. Cl. C40B 30/04; A61K 31/513; A61P 35/00; A61K 31/282; A61K 33/24; C40B 40/06; A61K 31/505

(52) U.S. Cl. 424/649; 506/9; 506/16; 514/274; 514/256; 514/492

(57) ABSTRACT

The invention relates to methods for diagnosis and prognosis of gastric cancer. The approach described herein can distinguish intestinal-type gastric cancer (G-INT) from diffuse-type gastric cancer (G-DIF). The genomic expression signatures of G-INT and G-DIF define two major sets of genes. A diagnosis of gastric cancer G-INT and G-DIF can be made on the basis of the expression levels of these genes. This can lead to a better prognosis and treatment of gastric cancer.

### CROSS-REFERENCE TO RELATED APPLICATION

This application claims benefit of, and priority from, U.S. provisional patent application No. 61/476,698, filed on Apr. 18, 2011, the contents of which are fully incorporated herein by reference.

### FIELD OF THE INVENTION

The invention relates to diagnosis, prognosis and treatment of gastric cancer.

### BACKGROUND

Gastric adenocarcinoma (gastric cancer, GC) is the second leading cause of global cancer mortality and 4th most common cancer worldwide. Most GC patients present with late stage disease with an overall 5-year survival of about 20%. A wealth of clinical, molecular, and pathological data suggests that GC is a heterogeneous disease. Objective response rates to conventional chemotherapeutic regimens range from 20-40%, indicating that individual GCs can exhibit a range of responses when treated identically. Canonical oncogenic pathways such as E2F, K-RAS, p53, and Wnt/β-catenin signalling are also known to be deregulated with varying frequencies in GC, suggesting a high degree of molecular heterogeneity. However, despite evidence that GCs can exhibit striking inter-individual differences in disease aggressiveness, histopathologic features, and responses to therapy, most GC patients today are managed alike with a “one size fits all” approach resulting in markedly diverse clinical outcomes. Approaches capable of classifying heterogeneous populations of GC patients into biologically and clinically homogenous subgroups are thus urgently required, such that GC patient prognoses can be accurately predicted, and clinical decisions made based on the underlying biology of each subgroup.

Reflecting this urgency, several classification systems for GC have been reported over the decades. In 1965, Lauren described two main subtypes of GC, intestinal (G-INT) and diffuse (G-DIF), on the basis of microscopic features observed in gastric tumors (Lauren P., Acta Pathol Microbiol Scand, 1965, 64:31-49). But note that while the intestinal and diffuse subtypes are correlated with G-INT and G-DIF, about 30% of cases are discordant. Thus Lauren's classification and G-INT/G-DIF should not be regarded as the same. Since then, several other GC histopathological classifications have since been developed, such as the systems of the WHO (Jass J. R. et al., Cancer, 1990, 66:2162-7); Ming S. C., Cancer, 1977, 39:2475-85; Mulligan R. M., Pathol Annu, 1972, 7:349-415; and Goseki N. et al., Gut, 1992, 33:606-12, and more recently, molecular classifications based on immunohistochemistry, gene expression profiles (Kim B. et al., Cancer Res, 2003, 63:8248-5518-20; Vecchi M. et al., Oncogene, 2007, 26:4284-94; and Boussioutas A. et al., Cancer Res, 2003, 63:2569-77), proteomics (Lee H. S. et al., Clin Cancer Res, 2007, 13:4154-63), and integrative systems biology approaches (Aggarwal A. et al., Cancer Res, 2006, 66:232-41; Tay S. T. et al., Cancer Res, 2003, 63:3309-16; Myllykangas S. et al., Int J Cancer, 2008, 123:817-25). However, to date, none of these GC classification systems been shown to provide reliable independent prognostic information, nor have they been able to suggest specific treatment options for patients.

One common feature shared by most previously-described GC classification systems is that they have principally focused on the characterization of primary tumors, which are known to contain many distinct cell types including tumor cells, fibroblastic/desmoplastic stroma, blood vessels, and immune cells.

There remains a need for a clinically meaningful GC taxonomy to classify GC and to provide prognostic and predictive value.

### SUMMARY

The invention relates to methods for diagnosis and prognosis of gastric cancer. The approach described herein aims to distinguish intestinal-type gastric cancer (G-INT) from diffuse-type gastric cancer (G-DIF). The genomic expression signatures as disclosed herein define two major sets of genes. It is submitted that a diagnosis of gastric cancer G-INT and G-DIF can be made on the basis of the expression levels of these genes. This can lead to a better prognosis and treatment of gastric cancer.

In one aspect, the invention relates to a method of diagnosing intestinal-type gastric cancer (G-INT). The method comprises the step of determining the expression levels of the following Group A1 genes in gastric tissue in a biological sample from a subject having gastric cancer: TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2 and TMC5. In addition, the expression level of at least one of the following Group A2 genes in the biological sample may also be determined for greater accuracy and precision: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 and HEPH. An increase in the expression levels of the Group A1 and optional Group A2 genes in the subject, in comparison with expression levels of the genes in non-cancerous gastric tissue, would indicate that the subject has G-INT.

A further aspect of the invention relates to a method of diagnosing diffuse-type gastric cancer (G-DIF). The method comprises determining the expression levels of the following Group B1 genes in gastric tissue in a biological sample from a subject having gastric cancer: RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586 and RASSF8. In addition, the expression level of at least one of the following Group B2 genes in the biological sample may also be determined for greater accuracy and precision: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 and MAP1B. An increase in the expression levels of the Group B1 and optional Group B2 genes in the subject, in comparison with expression levels of the genes in non-cancerous gastric tissue, would indicate that the subject has G-DIF.

In accordance with another aspect of the invention, there is provided a method of diagnosing G-INT by RNA analysis. The method comprises steps such as: obtaining a gastric tissue sample from a subject with gastric cancer; isolating RNAs from the sample for a gene expression analysis; analyzing the RNAs by a hybridization analysis or a sequencing analysis to determine the expression levels of the following Group A1 genes in the sample: TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2 and TMC5, and optionally determining the expression level of at least one of the following Group A2 genes in the sample: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 and HEPH, wherein higher expression levels of the Group A1 and optional Group A2 genes in the subject, compared to expression levels of the genes in non-cancerous gastric tissue, indicates that the subject has G-INT.

In accordance with another aspect of the invention, there is provided a method of diagnosing G-DIF by RNA analysis. The method comprises steps such as: obtaining a gastric tissue sample from a subject with gastric cancer; isolating RNAs from the sample for a gene expression analysis; analyzing the RNAs by a hybridization analysis or a sequencing analysis to determine the expression levels of the following Group B1 genes in the sample: RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586 and RASSF8, and optionally determining the expression level of at least one of the following Group B2 genes in the sample: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 and MAP1B, wherein higher expression levels of the Group B1 and optional Group B2 genes in the subject, compared to expression levels of the genes in non-cancerous gastric tissue, indicates that the subject has G-DIF.

In certain aspects of the invention, the hybridization analysis comprises a microarray analysis. In certain aspects, the microarray analysis uses commercially available microarrays such as an Affymetrix Human Genome U133 Plus 2.0 array or an Affymetrix U1333AB array. In other aspects, the hybridization analysis comprises a microarray analysis using an Illumina Human-6 v2 Expression Beadchips. In other aspects, the hybridization analysis comprises a customized array comprising probes for detection of the genes of the methods described herein.

In other aspects of the invention, the hybridization analysis comprises a real-time polymerase chain reaction with detection of amplification of genes by fluorescent probes.

In certain aspects of the invention, the sequencing analysis comprises a high-throughput sequencing analysis. In certain aspects, the high-throughput sequencing methods include, but are not limited to SOLiD sequencing, 454 sequencing and Solexa sequencing. In certain aspects, the high-throughput sequencing methods are used in conjunction with SAGE or superSAGE for the gene expression analysis.

In certain aspects of the invention, the gene expression analysis comprises a comparative genomic hybridization assay. In some embodiments, this assay includes detection by epifluorescence microscopy.

In accordance with another aspect of the invention, there is provided a method of diagnosing G-INT by protein analysis. The method comprises steps such as: obtaining a gastric tissue sample from a subject with gastric cancer; isolating proteins from the sample for a gene expression analysis; analyzing the proteins by a protein affinity-based method or by a mass spectrometry-based proteomics method to determine the levels of proteins encoded by the following Group A1 genes in the sample: TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2 and TMC5, and optionally determining the expression level of at least one of the following Group A2 genes in the sample: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 and HEPH, wherein higher expression levels of the Group A1 and optional Group A2 genes in the subject, compared to expression levels of the genes in non-cancerous gastric tissue, indicates that the subject has G-INT;

In accordance with another aspect of the invention, there is provided a method of diagnosing G-DIF by protein analysis. The method comprises steps such as: obtaining a gastric tissue sample from a subject with gastric cancer; isolating proteins from the sample for a gene expression analysis; analyzing the proteins by a protein affinity-based method or by a mass spectrometry-based proteomics method to determine the expression levels of the following Group B1 genes in the sample: RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586 and RASSF8, and optionally determining the expression level of at least one of the following Group B2 genes in the sample: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 and MAP1B, wherein higher expression levels of the Group B1 and optional Group B2 genes in the subject, compared to expression levels of the genes in non-cancerous gastric tissue, indicates that the subject has G-DIF.

In certain aspects of the invention, the protein affinity method comprises detection of specific proteins using interactions with antibodies or antibody fragments. The interactions may be provided by antibodies or antibody fragments. The antibodies or antibody fragments may be deposited on an antibody microarray.

In other aspects of the invention, the mass-spectrometry-based proteomics method uses Fourier Transform electrospray ionization mass spectrometry or matrix-assisted laser ionization/desorption mass spectrometry.

In one aspect of the invention, the mass-spectrometry-based proteomics analysis method is APEX.

A further aspect of the invention relates to a method for prognosis of gastric cancer in a subject. The method comprises the steps of determining the expression levels of the Group A1 genes and Group B1 genes as defined above, in gastric tissue in a biological sample from a subject having gastric cancer, and optionally determining the expression level of at least one of the Group A2 genes and Group B2 as defined above. Compared to expression levels of the genes in non-cancerous gastric tissue, an increase in the expression levels of the Group A1 and optional Group A2 genes would indicate that the subject has G-INT. Similarly, an increase in the expression levels of the Group B1 and optional Group B2 genes would indicate that the subject has G-DIF. Information about whether the subject has G-INT or G-DIF would be of prognostic value.

A further aspect of the invention relates to a method of treating gastric cancer in a subject. The method comprises determining whether the subject has intestinal-type gastric cancer (G-INT) or diffuse-type gastric cancer (G-DIF) by determining the expression levels of the Group A1 genes in gastric tissue in a biological sample from a subject having gastric cancer, and optionally determining the expression level of at least one of the Group A2 genes; and determining the expression levels of the Group B1 genes from the same subject, and optionally determining the expression level of at least one of the Group B2 genes. Then, guided by the results, chemotherapeutic treatment may be designed for the subject, taking into account the likelihood that the subject has G-INT or G-DIF. If the subject has G-INT, administering 5-fluorouracil or a fluoropyrimidine, and/or oxaliplatin to the subject may be appropriate. If the subject has G-DIF, administering cisplatin as an example may be appropriate.

A further aspect of the invention relates to an array comprising a set of polynucleotide probes. The set of polynucleotide probes are specific for the expression products of the Group A1 genes as defined above, and optionally at least one of the Group A2 genes as defined above. Alternatively, the set of polynucleotide probes are specific for the expression products of the Group B1 genes defined above, and optionally at least one of the Group B2 genes as defined above. It is contemplated that the set of polynucleotide probes are specific to the genes associated with gastric cancer, i.e. the Groups A1, A2, B1 and B2 genes, and does not include irrelevant genes. The array can comprise the set of polynucleotides specific for the expression products of the Group A1 genes and the Group B1 genes.

### BRIEF DESCRIPTION OF THE DRAWINGS

In drawings illustrating embodiments of the invention:

FIG. 1 shows that unsupervised clustering of gastric cancer cell lines (GCCL) reveals 2 major intrinsic subtypes. (A) Hierarchical dendrogram depicting clustering of 37 GCCLs into G-INT (left branches) and GDIF (right branches); height: squared euclidean distances between cluster means. (B) Silhouette widths of individual cell lines when classified in 2 clusters. Silhouette width: a measure for each sample of membership of within its own class against that of another class. (C) heat map of expression of 171 genes obtained from microarray data using linear models for microarray data (LIMMA) arranged by hierarchal clustering of cell lines (columns) and expression difference for each gene between G-INT and G-DIF as measured by the t-test statistic (rows).

FIG. 2 shows associations of intrinsic subtypes with Lauren's classification in primary GCs. Heat map of gene expression in (A) SG and (B) AU cohorts arranged by strength of association (columns) and expression difference for each gene between G-INT and G-DIF as measured by the t-test statistic (rows). 1st row label shows Laurens class; 2nd row label shows intrinsic classes (G-INT or G-DIF). Representative hematoxylin and eosin (H & E) section of (C) G-INT/intestinal cancer and (D) G-DIF/Diffuse cancer. (E) Histogram showing that the 2 genomic subtypes were differentially enriched among Lauren's intestinal and diffuse histological subtypes (p<0.001, chi square test). The subclasses are therefore referred to as Genomic Intestinal and Genomic Diffuse.

FIG. 3 shows that intrinsic genomic subclasses are prognostic. Kaplan-Meier plots of survival in (A) all patients (HR: 1.79, 95% Cl: 1.28-2.51, p=0.001) and (B) when the intrinsic classification and Lauren's classes are discordant (HR 1.83, 95% Cl: 1.02-3.30, p=0.04). Note that whilst other published signatures are not prognostic, the intrinsic subtypes are prognostic. Intrinsic diffuse has inferior overall survival: 30 months vs. 71 months (HR: 1.48, 95% Cl: 1.14-1.192, p<0.01, univariate analysis and HR 1.39; 95% Cl: 1.05-1.78, p=0.02 after adjusting for stage. In multivariate analysis, intrinsic subtypes is prognostic, independent of stage and Lauren's histology.

FIG. 4 shows in vitro chemosensitivity of G-INT and G-DIF cell lines. GI-50 values of 11 G-INT and 17 G-DIF cell lines upon treatment with 5-FU, oxaliplatin and cisplatin. GI-50s refer to the drug concentration at which 50% growth inhibition is achieved. (y-axis: GI-50 enumerated in negative log 10). The horizontal lines represent the therapeutic concentration patients are exposed to based on pharmacokinetic data (Saif M. W. et al., J Natl Cancer Inst, 2009, 101:1543-52; Ikeda K. et al., Jpn J Clin Oncol, 1998, 28:168-75; Graham M. A. et al., Clin Cancer Res, 2000, 6:1205-18). Mean GI-50 concentrations for G-INT and G-DIF cell lines respectively: 5FU: 5.20 μM, 23.22 μM; Cisplatin: 38.61 μM, 13.35 μM; Oxaliplatin: 1.33 μM, 5.49 μM.

FIG. 5 shows PCA and NMF plots of 37 GC cell lines. (A) Principal component analysis (PCA) of 37 Gastric cancer cell lines. G-INT and G-DIF cell lines are distinguished by the first principal component. (B) Reordered consensus matrices. An average of 1000 connectivity matrices were computed at k=2-5 for the 37 gastric cell lines using the selected genes. Samples were hierarchically clustered using the consensus clustering matrix from 0 (squares, samples are never in the same cluster) to 1 (circles, samples are always in the same cluster). The y axis lists the cell line names. (C) Cophenetic correlation coefficient plot corresponding to k=2-7. A two-class decomposition is suggested.

FIG. 6 shows that G-INT/G-DIF is prognostic in the SG cohort and AU cohorts. Kaplan-Meier plots of survival in (A) SG cohorts (HR 1.78, 95% Cl: 1.19-2.64, p=0.004) and (B) AU cohort (HR 1.73, 95% Cl: 0.92-3.26, p=0.09). G-INT and G-DIF are prognostic.

FIG. 7 shows a tissue microarray dataset. (A) Representative immunostaining expression of CDH17 and LGALS4 in gastric cancer. (1,4) Positive membraneous CDH17 expression (2,5) Negative CDH17 expression (3,6) Positive cytoplasmic LGALS4 expression. (B) Kaplan-Meier plots of survival of tumors positive for both LGALS4 and CDH17 (2-marker positive) compared to tumors negative for both markers (2-marker negative) (HR 1.95, 95% Cl: 1.13-3.38, p=0.02, adjusted for stage).

### DETAILED DESCRIPTION OF EMBODIMENTS

Due to the high level of tissue complexity, subtle variations in diverse cell types, both across and within-tumors, can cause differences in interpretation between observers, and ultimately pose difficulties for standardization across different centres. The present invention provides an alternative strategy that initially focused not on primary GCs, but on a diverse panel of GC cell lines. Since cancer cell lines are devoid of other cell types such as fibroblasts, endothelial, and immune cells, any genomic differences detected in cell lines should be by nature tumor-centric and thereby “intrinsic” to the underlying biology of the GC cancer cell.

Investigation of a large panel of GC cell lines permitted us to identify a genomic expression signature clearly defining two major intrinsic subgroups of GC. These intrinsic subgroups were validated in primary tumors and, when applied to 4 independent GC cohorts, the intrinsic subtypes proved capable of providing independent prognostic information (see Example 5). In vitro and in vivo evidence also demonstrated that GCs belonging to different intrinsic subtypes may respond differently to various standard-of-care chemotherapies.

Unlike previous approaches for comparative molecular examination of GC (Jinawath N. et al., Oncogene, 2004, 23:6830-44; Wang L. et al., World J Gastroenterol, 2006, 12:6949-54; Meireles S. I. et al., Cancer Res, 2004, 64:1255-65), the method described herein used unsupervised approaches for subclass discovery. The present invention aims to address several deficiencies in approaches known in the art, namely a) the major distinctions in the molecular heterogeneity of GC might be unrelated to presently known classification systems or phenotypes, and b) using current classification systems, reproducibility among pathologists is only about 70% (Arslan C. et al., Histopathology 1982, 6:391-8; Dixon M. F. et al., Histopathology, 1994, 25:309-16; Palli D. et al., Br J Cancer, 1991, 63:765-8; Shibata A. et al., Cancer Epidemiol Biomarkers Prey, 2001, 10:75-8) and this lack of inter-observer concordance might compromise supervised analysis. Testing of several different prediction algorithms confirmed that the intrinsic subtypes exhibited stable and reproducible classification performance in cell lines and primary tumors, thus demonstrating that the subtypes are statistically robust.

Using a strict filtering criteria (FDR<0.002), a genomic classifier of 171 genes exhibiting differential regulation between the subtypes was identified. Biological curation of the classifier confirmed that the intrinsic subtypes are associated with very different gene expression features, cellular processes and biological pathways. These results demonstrate that the intrinsic subtypes are very distinct and may represent distinct lineages.

The clinical relevance of the intrinsic subclasses is supported by the finding that it can act as an independent predictor of clinical survival in multiple patient cohorts, even after controlling for tumor stage. Intestinal cancers are classically characterized by glandular differentiation on a background of gastric atrophy or intestinal metaplasia, while diffuse cancers typically appear as rows of single mononuclear “signet ring” cells with little cell adhesion. These apparently distinct features, however, are not always discernable in clinical samples where inter-observer variation and unclassifiable or “mixed” subtypes are not uncommonly reported. As described herein, patients stratified by Lauren's histopathology did not exhibit significantly different survival outcomes, while patients discordant between the intrinsic subclasses and Lauren's exhibited survival patterns that support the intrinsic genomic taxonomy. The present results show that the intrinsic subclasses provide information about the predominant lineage in GC samples that may not be precisely distinguished by morphology, and that this information is clinically relevant.

Besides gene expression, two genes in the classifier (LGALS4 and L1-Cadherin (CDH17)) were employed as immunohistochemical markers for the G-INT intrinsic subtype. LGALS4 and CDH17 have been previously reported to be differentially regulated across subsets of gastric tumors (Chen X. et al., Mol Biol Cell, 2003, 14:3208-15) and cell lines (Ji J. et al., Oncogene, 2002, 21:6549-56), and expressed in intestinal metaplasia (Dong W. et al., Dig Dis Sci, 2007, 52:536-42; Lee H. J., Gastroenterology, 2010, 139:213-25 e3). CDH17 was recently reported as a prognostic factor in early-stage GC (Lee H. J., Gastroenterology, 2010, 139:213-25 e3), a marker of poor prognosis in another study (Ito R. et al., Virchows Arch, 2005, 447:717-22), and a potential therapeutic target in experimental models (Liu Q. S. et al., Cancer Sci, 2010, 101:1807-12). The 2-marker positive group was specifically compared to the 2-marker negative group to confidently distinguish between the GINT and G-DIF cancers. Our results showed that the one-third of 1-marker positive patients also appeared to exhibit an improved survival trend compared to the 2-marker negative group (CDH17, p=0.08 adjusted for stage; LGALS4, p=0.07 adjusted for stage). These results show that some of the 1-marker positive cancers may also be G-INT cancers as well (FIGS. 8 A & B).

In vitro, G-INT lines were more sensitive to 5-FU and oxaliplatin than G-DIF cell lines, but were also more resistant to cisplatin. The absolute magnitude of these in vitro differential sensitivities is about 3-5 fold. A significant interaction between the intrinsic subtypes and differential benefit from adjuvant 5-FU therapy was observed in retrospective patient cohorts (Table 3 and Table 8). These results show that in addition to patient prognosis, the intrinsic subtypes can be used to guide treatment selection.

In INT-0116 (Macdonald J. S., J Clin Oncol, 2009, 27:abst 4515), a ten-year update subgroup analysis revealed that all GC subsets benefited from 5-FU therapy except for cases with diffuse histology. Moreover, in JCOG 9912 (Boku N. et al., Lancet Oncol, 2009, 10:1063-9) which established S-1 monotherapy as a first-line palliative chemotherapy option in Japan, benefit of irinotecan/cisplatin over 5-FU based monotherapy was observed in diffuse but not intestinal GCs. The results described herein are consistent with subgroup analysis of these two large GC clinical trials. Therefore, the intrinsic subtypes described herein provide a clinically relevant genomic taxonomy of GC with prognostic and predictive value.

The genomic expression signatures identified herein define two major intrinsic subgroups of GC which allows for differentiation between G-INT and G-DIF:

Intestinal-type gastric cancer (G-INT) involve the 92 gene(s) listed in Table 5 (referred to henceforth as “Group A”): TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2, TMC5, CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 or HEPH. Diffuse-type gastric cancer (G-DIF) involve the 79 gene(s) (referred to henceforth as “Group B”): RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586, RASSF8, NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 or MAP1B.

An increase in the expression level of the above gene(s) in the subject, compared to expression level of the corresponding gene(s) in non-cancerous gastric tissue, indicates that the subject probably has G-INT or G-DIF. Treatment of the subject for GC can be guided accordingly. It should be noted that although 92 genes are indicated for G-INT and 79 genes for G-DIF, not all these genes need to be assayed for expression in order to obtain a diagnostic or prognostic value for G-INT and G-DIF. The aim is to provide a minimum set of polynucleotides that would be useful in diagnosing G-INT or G-DIF. Any number of gene(s) from the above sets that permits diagnosis within acceptable diagnostic parameters is contemplated.

It is contemplated that the number of genes whose expression is to be assayed may be a few from the relevant set, or any number up to all of the genes identified in the relevant set. Specifically, it is contemplated, based on the analysis set forth in the Examples, that the group of 29 genes (referred to henceforth as “Group A1”): TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2 and TMC5, would be sufficient for the diagnosis or prognosis of G-INT. Determination of the expression level of at least one additional gene from the remainder of Group A should improve accuracy. It is contemplated that the expression levels of at least 1, 5, 10, or at least 20, at least 30, at least 40, at least 50, or all 63 remaining genes of Group A may be assayed.

For example, the additional genes from Group A can comprise at least one of or any combination of:

### CYP3A5, EPS8L3, FA2H, TOX3 and BAIAP2L2;

### PIP5K1B, AGPAT2, BCL2L15, TNFRSF11A and PLCH1;

### GPR35, ATP10B, TC2N, MMP28 and CYP3A5;

### LLGL2, CAPN10, TRNP1, SDCBP2 and MYB;

### ACSM3, REG4, CYP2C18, PRR15 and SGK493;

### HNF4G, TMEM45B, KLF5, UGT8 and RNF128;

### KCNE3, LOC100133019, DNAJC22, ST6GALNAC1 and CLRN3;

### GDF15, RNF43, KIAA0746, USH1C and CLDN2;

EHF, FOXA3, POF1B, LOC286208 and C9orf152;
GMDS, SLC22A18AS, C11orf9, LOC100131701 and TMPRSS4;
SLC37A1, PTK6, CEACAM5, SULT2B1 and LOC120376; and/or

### MST1R, ELF3, SLC26A9, SLC40A1, PTPRB, AGR2, GALNT12 and HEPH.

It is also contemplated, based on the analysis set forth in the Examples, that the group of 17 genes (referred to henceforth as “Group B1”): RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586 and RASSF8, would be sufficient for the diagnosis or prognosis of G-DIF. Determination of the expression level of at least one additional gene from the remainder of Group B should improve accuracy for G-DIF diagnosis and prognosis. It is contemplated that the expression levels of at least 1, 5, 10, or at least 20, at least 30, at least 40, at least 50, or all 62 remaining genes of Group B may be assayed.

For example, the additional genes from Group B can comprise at least one of or any combination of:

### NUAK1, TMEFF1, SCHIP1, TMEM136 and ZCCHC11;

### FAM101B, FAM127A, SIX4, DENND5A and TTC7B;

### ZNF512B, KIRREL, GNB4, FN1 and GJC1;

### GLIPR2, FJX1, DSE, ENAH and DNAH14;

### CALD1, GPRASP2, HEG-int, DLX1 and TIMP3;

### GLT8D4, LPHN2, PTPRS, FRMD6 and SNAP47;

### WHAMML1, WHAMML2, GATA2, APH1B and MLLT11;

### PPM1F, SNX21, ANXA6, PKIG and ANTXR1;

### ATP8B2, CSRP2, DEGS1, KLHDC8B and DEPDC1;

### CSE1L, WDR35, SAMD4A, TRIM23 and FAM92A1;

S1PR3, TUBA1A, LOC644450, PTPN1 and HOMER3; and/or

### IGFBP7, TSR1, AURKB, MSX1, CTSL1, TEAD1, LOC283658 and MAP1B.

For further accuracy and precision of gastric cancer prognosis, it is contemplated that the subsets of genes above which are sufficient indicators of G-INT and G-DIF, are both assayed for the same subject. For example, about 44 genes of the 171 genes, based on the results of the analysis in the Examples, to 46 genes (Group A1+Group B1) can be assayed.

Assays of non-relevant genes, i.e. other than the genes of Groups A and B, such as those provided in the Affymetrix DNA array or such arrays known in the art as research tools, are not intended to be included in the present invention. Thus it is contemplated that the expression levels of no other genes than the 171 genes of Groups A1, A2, B1 and B2 are determined.

As used herein, “gastric cancer” is intended to encompass, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc. “Metastatic disease” refers to cancer cells that have left the original tumor site and migrate to other parts of the body, for example via the bloodstream or lymph system. The two main subtypes of gastric cancer are described by Lauren, that is intestinal-type (G-INT) and diffuse-type (G-DIF) (Lauren P., Acta Pathol Microbiol Scand, 1965, 64:31-49, hereby incorporated by reference).

As used herein, “tissue” is intended to encompass a plurality of functionally related cells. A tissue can be a suspension, a semi-solid, or solid. Tissue includes cells collected from a subject, as well as cell lines grown ex vivo or in vitro.

As used herein, “diagnosing” or “diagnosis” is intended to encompass the process of identifying gastric cancer by its signs, symptoms and results of various tests. Diagnosing gastric cancer includes the methods described herein. In one embodiment, diagnosing gastric cancer includes determining whether a subject likely has intestinal-type gastric cancer (G-INT) or diffuse-type gastric cancer (G-DIF). This determination may help in choosing an appropriate course of treatment with a greater chance of success.

As used herein, “expression” of a gene is intended to encompass the process by which the coded information of a gene is converted into an operational, non-operational, or structural part of a cell, such as the synthesis of a protein. When used in reference to the expression of a nucleic acid molecule, such as a gene, an increase in the expression level of a gene refers to any process which results in an increase in production of a gene product. A gene product can be RNA (such as mRNA, rRNA, tRNA, and structural RNA) or protein. Therefore, an increase in the expression level of a gene includes processes that increase transcription of a gene or translation of mRNA. The “expression level” of a nucleic acid molecule in a cancerous cell or tissue can be altered relative to a non-cancerous or normal (wild type) cell or tissue. Alterations in the expression of a nucleic acid molecule is associated with a change in expression of the corresponding or RNA protein. The change can result in an increase or decrease of the expression product. In certain embodiments, an increase in expression of the relevant set of genes indicate that the gastric cancer is likely to be G-INT or G-DIF. Controls or standards for comparison to a sample, for the determination of differential expression, include samples believed to be normal, for example, a sample such as gastric tissue from a subject that does not have gastric cancer.

An increase in the expression level of a gene includes any detectable increase in the production of a gene product. In certain examples, production of a gene product (such as those listed in Table 5) increases by at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, or at least 4-fold, as compared to expression level of the gene in non-cancerous tissue which may be gastric tissue.

As is clear from the description above, an expression level of gene can be “determined” using any method available in the art. A variety of methods may be used which involve analysis of nucleic acids and proteins. Traditional methods for analysis of nucleic acids and proteins include Northern blots for analyzing RNA and Western blots for analyzing proteins. The newer techniques described hereinbelow are better suited for high throughput analyses of gene expression levels in most cases.

Nucleic acid-based methods may be based on detection and/or characterization of an mRNA product of the genes of interest. Such nucleic acid-based analysis methods include nucleic acid hybridization-based methods and nucleic acid sequencing methods. These methods require isolation of RNA. A number of commercially-available kits such as the RNeasy purification kits (www.qiagen.com), NucleoSpin RNA columns (www.clontech.com), and GeneJet RNA purification kits, for example are available for this purpose. RNA isolated by such kits can be then used in the methods described herein. In some cases, platform manufacturers will have one or more recommended kits selected for platform compatibility.

Protein-based analyses appropriate for use in the methods described herein include protein affinity detection methods and mass-spectrometry proteomics analysis methods. Processes for purifying proteins for protein-based analyses tend to be more complicated than the processes used to purify RNA and may include a number of chromatographic separation methods, such as size exclusion chromatography, ion exchange chromatography, reversed phase chromatography and affinity chromatography, as well as electrophoretic methods. The uses of these techniques will depend upon the platform used for the subsequent analyses. Furthermore, evaluation of the purified proteins may be needed prior to initiating gene expression analyses. Exemplary methods and techniques for preparing proteins for proteomics analyses can be found, for example, in Purifying Proteins for Proteomics—A Laboratory Manual, 2004, Cold Spring Harbor Press, Richard J. Simpson ed., which is incorporated herein by reference.

In terms of nucleic acid hybridization methods, gene expression analysis is generally performed using a nucleic acid probe for measuring the level of mRNA (or a cDNA corresponding to the mRNA), to which the probe has been engineered to bind, where the probe binds the intended species and provides a distinguishable signal. Exemplary methods for selecting PCR primers and/or hybridization probes are included in Innis et al., eds., 1990, PCR Protocols: A Guide to Methods and Applications, Academic Press Inc., San Diego, Calif.; Froehler et al., 1986, Nucleic Acid Res. 14:5399-5407; McBride et al., 1983, Tetrahedron Lett. 24:246-248, U.S. Pat. No. 7,013,221, each of which is incorporated by reference. Probes usually have lengths of at least 20 nucleotides to provide requisite specificity for detecting expression, although they may be shorter depending upon other species expected to be found in sample.

In some embodiments, a set of nucleic acid probes capable of hybridizing to RNA or cDNA allows quantification of the expression level and prediction of the clinical outcome based on this quantification. In some embodiments, the probes are affixed to a solid support, such as a microarray. Microarrays are described in more detail hereinbelow.

In other embodiments the real time polymerase chain reaction (also known as quantitative PCR(qPCR)) may be used as a hybridization-based method which allows amplified DNA corresponding to the genes of interest to be detected in real time as the amplification reaction progresses. This method requires that the RNA of interest, such as transcribed mRNA be first transcribed to cDNA using reverse transcriptase before amplification begins. Two common methods for detection of products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target. The physical properties of such dyes and reporters provide the physical characteristics required for quantitation of gene expression in the methods described herein.

Another technique which may be used in the methods described herein is comparative genomic hybridization (CGH). In this technique, DNA samples from subject tissue and from normal control tissue are labeled with different tags for later analysis by fluorescence. After mixing subject and reference DNA along with unlabeled human cot-1 DNA (placental DNA that is enriched for repetitive DNA sequences such as the Alu and Kpn family) to suppress repetitive DNA sequences, the mixture is hybridized to normal metaphase chromosomes or, in the case of array- or matrix-based CGH, to a slide containing hundreds or thousands of defined DNA probes. Using epifluorescence microscopy and quantitative image analysis, regional differences in the fluorescence ratio of gains/losses vs. control DNA can be detected and used for identifying abnormal regions in the genome. CGH is described in detail in U.S. Pat. No. 6,335,167, which is incorporated herein by reference in entirety.

High-throughput nucleic acid sequencing, which is also known to those skilled in the art as “next-generation sequencing” may be used in certain embodiments of the methods described herein. Examples of high throughput sequencing include massively parallel signature sequencing (MPSS) developed by Lynx Therapeutics, (Zhou et al, Methods Mol. Biol. 2006; 331: 285-311, incorporated herein by reference in entirety); the SOLiD platform of Applied Biosciences Inc. (www.appliedbiosystems.com), the pyrosequencing platform developed by 454 Life Sciences (now Roche Diagnostics Inc., www.roche.com/diagnostics/), and Solexa sequencing (Illumina Inc., www.illumina.com), among others.

Next-generation sequencing is particularly powerful in context of the methods described herein when combined with a technique known as superSAGE, a variation of SAGE (serial analysis of gene expression) (see for example, Matsumura et al., Proc. Natl. Acad. Sci. USA 100, 26: 15718, incorporated herein by reference in entirety). In the original SAGE method, mRNA is isolated and a portion of the sequence is extracted from a defined position from each mRNA molecule. The portions are then linked into a long chain or concatemer and cloned into a vector for transfection of bacteria to obtain high copy numbers. The concatemers are then sequenced using modern high throughput methods and the data are processed to count the sequence portions.

SuperSAGE uses the type III-endonuclease EcoP15I of phage P1, to cut 26 bp long sequence tags from cDNA corresponding to each mRNA transcript, expanding the tag-size by at least 6 bp relative to the predecessor techniques SAGE and LongSAGE. The longer tag size allows for a more precise allocation of the tag to the corresponding transcript, because each additional base increases the precision of the annotation considerably. By direct sequencing with modern next-generation sequencing techniques, hundreds of thousands or millions of tags can be analyzed simultaneously, producing very precise and quantitative gene expression profiles. Therefore, this method can provide accurate transcription profiles.

Measurements of proteins for determining protein expression levels can be accomplished by using a specific binding reagent, such as an antibody. One of ordinary skill in the art would recognize that different affinity reagents could be used with present invention, such as one or more antibodies (e.g., monoclonal or polyclonal antibodies) and the invention can include using techniques such as ELISA for the analysis.

Specific antibodies (e.g., specific to the genes of the proteins encoded by the genes of interest) can be used in methods described herein for gene expression analysis. Antibodies and related affinity reagents such as, e.g., antibody fragments, and engineered sequences such as single chain Fvs (scFvs) must specifically bind their intended target, i.e., a protein encoded by a gene included in the molecular signature of interest. Specific binding includes binding primarily or exclusively to an intended target.

Antibodies can be identified and obtained from a variety of sources, such as the MSRS catalog of antibodies (Aerie Corporation, Birmingham, Mich.), or can be prepared via conventional antibody-generation methods. Methods for preparation of polyclonal antisera are taught in, for example, Ausubel, F. M. et al., Current Protocols in Molecular Biology, Volume 2, 1997, pp. 11.12.1-11.12.9 (incorporated by reference). Preparation of monoclonal antibodies is taught for example, in Ausubel, F. M. et al., Current Protocols in Molecular Biology, Volume 2, 1997, pp. 11.4.1-11.11.5 (incorporated by reference in entirety). Preparation of scFvs is taught in, e.g., U.S. Pat. Nos. 5,516,637 and 5,872,215, both of which are incorporated by reference in their entirety.

Antibody arrays can be used in conjunction with the methods described herein. As described by Walter et al, Curr. Opin. Microbiol. 2000, 3: 298-302, (and references contained therein, each of which is incorporated herein by reference in entirety), an attractive method for fabricating antibody arrays involves the use of a micromolded hydrogel stamper and an aminosilylated receiving surface. The stamper deposits protein (e.g. antibody) as a submonolayer, as shown by I¹²⁵ labelling and atomic force microscopy. This allows antibody activity to be retained. Other approaches described by Walters et al., for preparation of protein microarrays involve using either photolithography of silane monolayers or gold, combining microwells with microsphere sensors, or inkjetting onto polystyrene film. These advances focus on the fabrication of miniaturized immunoassay formats by arraying of single proteins such as monoclonal antibodies.

Also in terms of protein analyses, mass spectrometry-based proteomics methods may be used in the methods described herein. Such methods use matrix-assisted laser desorption/ionization (MALDI) or electrospray ionization (ESI) mass spectrometric characterization of proteins. Adaptations of mass spectrometry-based proteomics methods for gene expression analysis are reviewed, for example, in Pasa-Tolic et al., J. Mass Spectrom. 2002, 37: 1185-1198, which is incorporated herein by reference in entirety.

In one exemplary technique for gene expression profiling, known as APEX (Lu et al., Nature Biotech. 2007, 25: 117), proteins are analyzed using standard shotgun proteomics methods, beginning with tryptic digest of a protein mixture, liquid chromatographic separation of the mixture (2D HPLC), analysis of peptide masses by electrospray ionization mass spectrometry (MS), fragmentation of peptides and subsequent analysis of the fragmentation spectra (MS/MS). The method enables the number of peptides observed per protein to provide an estimate of the abundance of the proteins of interest, thereby quantitating the expression products. Mass spectrometry-based proteomics analysis methods such as APEX can be adapted for gene expression profiling tasks according to the methods described herein without undue experimentation.

As used herein, “biological sample” is intended to encompass a biological specimen containing genomic DNA, RNA (including mRNA), protein, or combinations thereof, obtained from a subject. Examples include, but are not limited to, tissue biopsy, surgical specimen, and autopsy material, or any material from the body which shows the same gene expression profile as gastric tissue. In one example, a sample includes a gastric cancer tissue biopsy.

In a particular embodiment, the gastric tissue biopsy is obtained endoscopically. The gastric tissue biopsy can be processed by a variety of acceptable methods known in the art. For example, the gastric tissue biopsy is placed immediately in RNAlater solution upon obtaining it from a subject. Total RNA is then extracted using any known methods and kits such as the Qiagen RNeasy Mini-kit (Qiagen) according to the instructions of the manufacturer. For the profiling, mRNAs may be hybridized to the probes specific for the sets of relevant genes described herein, preferably on a DNA array, according to techniques described herein as well as those known in the art.

The ability to differentiate between G-INT and G-DIF using the methods of the invention allows for cancer treatment that is directed specifically for treating G-INT or G-DIF by administering a chemotherapeutic agent to the subject in a manner most effective for the treatment of G-INT or G-DIF. In one aspect, once the subject is diagnosed as having intestinal-type gastric cancer, 5-fluorouracil or a fluoropyrimidine, and/or oxaliplatin, or any treatment that is effective for treating G-INT can be administered to the subject. In a further aspect, once the subject is diagnosed as having diffuse-type gastric cancer (G-DIF), cisplatin or any treatment that is effective for treating G-DIF can be administered to the subject.

As used herein, “treating” or “treatment” of gastric cancer is intended to encompass a therapeutic intervention that ameliorates a sign or symptom of a gastric cancer including, but not limited to, indigestion, loss of appetite, abdominal discomfort, abdominal irritation, abdominal pain, weakness, fatigue, bloating of the stomach, usually after meals, nausea, vomiting, diarrhea, constipation, weight loss, bleeding, anemia and dysphagia. Treatment can also induce remission or cure of gastric cancer. In particular examples, treatment includes prevention of gastric cancer, for example by inhibiting the full development or metastasis of a tumor. Prevention of gastric cancer does not require a total absence of disease. For example, a decrease of at least about 10%, at least about 20%, at least about 30%, at least about 40% or at least 50% can be sufficient. As contemplated herein, the treatment of gastric cancer encompasses treatments known in the art.

As used herein, “administration” or “administering” is intended to encompass providing or giving a subject an agent, such as a chemotherapeutic agent, by any effective route, including, but not limited to, injection (such as subcutaneous, intramuscular, intradermal, intraperitoneal, and intravenous), oral, sublingual, rectal, transdermal, intranasal, vaginal and inhalation routes.

As used herein, “chemotherapeutic agent” is intended to encompass any chemical agent with therapeutic usefulness in the treatment of gastric cancer. Examples of chemotherapeutic agents are known in the art (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Exemplary chemotherapeutic agents used for treating gastric cancer include carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, 5-fluorouracil or an oral fluoropyrimidine, oxaliplatin, mitoxantrone and vinorelbine. Combination chemotherapy is the administration of more than one chemotherapeutic agent to treat cancer. In one embodiment, the chemotherapeutic agent is 5-fluorouracil or a fluoropyrimidine, and/or oxaliplatin.

As used herein, “fluoropyrimidine” is intended to encompass oral fluoropyrimidines including capecitabine, tegafur/ftorafur, S-1, UFT (uracil/ftorafur, an oral agent with combines uracil, a competitive inhibitor of DPD, with the 5-FU prodrug tegafur) or UFT plus oral leucovorin or with folinic acid. S-1 is an orally active combination of tegafur which is a prodrug that is converted by cells to fluorouracil, gimeracil which is an inhibitor of dihydropyrimidine dehydrogenase (DPD) and degrades fluorouracil, and oteracil which inhibits the phosphorylation of fluorouracil in the gastrointestinal tract, thereby reducing the gastrointestinal toxic effects of fluorouracil. An alternative S-1 combination is S-1 (BMS 247616) which is composed of tegafur plus two modulators: a DPD inhibitor (5-chloro-2,4-dihydroxypyridine [CDHP]), and oxonic acid, an inhibitor of phosphoribosyl pyrophosphate transferase (an enzyme located in the gastrointestinal tract that causes decreased 5-FU incorporation into cellular RNA).

The chemotherapeutic agents 5-fluorouracil, oral fluoropyrimidines and/or oxaliplatin are preferred for treating intestinal-type gastric cancer. In another embodiment, the chemotherapeutic agent is cisplatin. The chemotherapeutic agent cisplatin is preferred for treating diffuse-type gastric cancer.

Methods for diagnosis of gastric cancer may involve the use of arrays. Both DNA arrays and protein arrays are contemplated.

In one aspect, the array comprises polynucleotides that hybridize to a subset of the genes listed in Table 5 G-INT involves the subset of 92 gene(s) listed in Table 5 (Group A, defined above). G-DIF involve the 79 gene(s) (Group B, defined above).

It is contemplated that the number of genes being probed on the array may be a few from the relevant set, or any number up to all of the genes identified in the relevant set. Specifically, it is contemplated, based on the analysis set forth in the Examples, that the group of 29 genes of Group A1 as defined above, would be sufficient in an array for the diagnosis or prognosis of G-INT. Inclusion of at least one additional gene on the array from the remainder of Group A should improve accuracy. It is contemplated that the array can include probes specific for at least 10, at least 20, at least 30, at least 40, at least 50, or all 63 remaining genes of Group A.

For example, the array may additionally include probes for at least one of or any combination of the following genes from Group A:

### CYP3A5, EPS8L3, FA2H, TOX3 and BAIAP2L2;

### PIP5K1B, AGPAT2, BCL2L15, TNFRSF11A and PLCH1;

### GPR35, ATP10B, TC2N, MMP28 and CYP3A5;

### LLGL2, CAPN10, TRNP1, SDCBP2 and MYB;

### ACSM3, REG4, CYP2C18, PRR15 and SGK493;

### HNF4G, TMEM45B, KLF5, UGT8 and RNF128;

### KCNE3, LOC100133019, DNAJC22, ST6GALNAC1 and CLRN3;

### GDF15, RNF43, KIAA0746, USH1C and CLDN2;

EHF, FOXA3, POF1B, LOC286208 and C9orf152;
GMDS, SLC22A18AS, C11orf9, LOC100131701 and TMPRSS4;
SLC37A1, PTK6, CEACAM5, SULT2B1 and LOC120376; and/or

### MST1R, ELF3, SLC26A9, SLC40A1, PTPRB, AGR2, GALNT12 and HEPH.

With respect to GC-DIF, it is contemplated, based on the analysis set forth in the Examples, that the group of 17 genes of Group B1 as defined above, would be sufficient in an array. Inclusion of at least one additional gene on the array from the remainder of Group B should improve accuracy. It is contemplated that the array can include probes specific for at least 1, 5, 10, or at least 20, at least 30, at least 40, at least 50, or all 62 remaining genes of Group B.

For example, the array may additionally include probes for at least one of or any combination of the following genes from Group B:

### NUAK1, TMEFF1, SCHIP1, TMEM136 and ZCCHC11;

### FAM101B, FAM127A, SIX4, DENND5A and TTC7B;

### ZNF512B, KIRREL, GNB4, FN1 and GJC1;

### GLIPR2, FJX1, DSE, ENAH and DNAH14;

### CALD1, GPRASP2, HEG-int, DLX1 and TIMP3;

### GLT8D4, LPHN2, PTPRS, FRMD6 and SNAP47;

### WHAMML1, WHAMML2, GATA2, APH1B and MLLT11;

### PPM1F, SNX21, ANXA6, PKIG and ANTXR1;

### ATP8B2, CSRP2, DEGS1, KLHDC8B and DEPDC1;

### CSE1L, WDR35, SAMD4A, TRIM23 and FAM92A1;

S1PR3, TUBA1A, LOC644450, PTPN1 and HOMER3; and/or

### IGFBP7, TSR1, AURKB, MSX1, CTSL1, TEAD1, LOC283658 and MAP1B.

For further accuracy and precision of gastric cancer prognosis, it is contemplated that the array would include both subsets of genes above which are sufficient indicators of G-INT and G-DIF. For example, the array can include oligonucleotides for about 44 genes of the 171 genes, based on the results of the analysis in the Examples, to all 46 genes of Group A1 and Group B1.

The specific arrays of the invention relate to the sets of genes associated with gastric cancer and are not intended to encompass commercially available microarrays such as a Affymetrix Human Genome U133 plus 2.0 Genechip or an Illumina Human-6 v2 Expression Beadchip, although the general construction of the array may be similar. Accordingly, one aspect of the invention involves determining the level of expression of no more than the sets of genes associated with G-INT or G-DIF, as disclosed herein; that is, it is contemplated that the arrays of the invention include probes for no other genes than the Groups A1, A2, B1 and B2 genes.

DNA microarray technology is known in the art and generally involves an arrayed series of DNA oligonucleotides (probes or reporters) used to hybridize a cDNA or cRNA sample (target) under high-stringency conditions. In a standard microarray, the probes are attached via surface engineering to a solid surface by a covalent bond to a chemical matrix (via epoxy-silane, amino-silane, lysine, polyacrylamide or others). The solid surface can be glass or a silicon chip.

As used herein, “array” is intended to encompass an arrangement of molecules, such as biological macromolecules (such as peptides or nucleic acid molecules) or biological samples (such as tissue sections), in addressable locations on or in a substrate. Arrays are also known as DNA chips or biochips. A “microarray” is an array that is miniaturized so as to require or be aided by microscopic examination for evaluation or analysis.

The array of molecules makes it possible to carry out a very large number of analyses on a sample at one time. In certain exemplary arrays, one or more molecules (such as an oligonucleotide probe) will occur on the array a plurality of times (such as twice), for instance to provide internal controls. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect expression of gastric-cancer-associated molecule sequences, such as at least one of those of the sequences listed in Table 5, such as at least 17, at least 29, at least 46, at least 50, at least 60, at least 75, at least 80, at least 90, at least 100, at least 150, or at least 171 sequences listed in Table 5 (for example, oligonucleotides for the 17 genes of Group B1, or for the 29 genes of Group A1, and optionally 1, 2, 3, 4, 5, 6, 7, 8, 9, 10, 11, 12, 13, 14, 15, 16, 17, 18, 19, 20, 25, 30, 35, 40, 44, 45, 50, 55, 60, 65, 70, 75, 80, 85, 90, 95, 100, 105, 110, 120, 130, 140, 150 or 171 of the remaining genes listed in Groups A and B). These are referred to collectively as oligonucleotide probes that are specific for the gastric cancer-associated genes.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including nucleic acids to which proteins are bound, or vice versa. In some examples, an array contains antibodies to gastric-cancer-associated proteins, such as any combination of proteins encoded by the sequences listed in Table 5, such as at least 17, at least 29, at least 46, at least 50, at least 60, at least 75, at least 80, at least 90, at least 100, at least 150, or at least 171 sequences listed in Table 5 (for example, protein probes for the 17 genes of Group B1, or for the 29 genes of Group A1, and optionally 1, 2, 3, 4, 5, 6, 7, 8, 9, 10, 11, 12, 13, 14, 15, 16, 17, 18, 19, 20, 25, 30, 35, 40, 44, 45, 50, 55, 60, 65, 70, 75, 80, 85, 90, 95, 100, 105, 110, 120, 130, 140, 150 or 171 of the proteins encoded by the remaining genes listed in Groups A and B).

As used herein, “polynucleotide” and “oligonucleotide” refers to nucleic acid molecules representing genes, for example DNA (intron or exon or both), cDNA, or RNA (such as mRNA), of any length suitable for use in detection, as a probe or other indicator molecule, and that is informative about the corresponding gene, such as those listed in Table 5. Nucleic acid molecules means a deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, a nucleic acid molecule can be circular or linear. Polynucleotide includes nucleic acid molecule analogs that function similarly to polynucleotides but which have non-naturally occurring portions. For example, polynucleotide analogs can contain non-naturally occurring portions, such as altered sugar moieties or inter-sugar linkages, such as a phosphorothioate oligodeoxynucleotide.

Particular polynucleotides can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides. In one example, a polynucleotide is a short sequence of nucleotides of at least one of the disclosed gastric-cancer-associated molecules listed in Table 5.

As used herein, “hybridizes to” or “hybridization” is intended to encompass formation of base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). It is intended that oligonucleotide probes hybridize under sufficiently stringent conditions such that the probes are specific for the expression products of the gastric cancer-associated genes.

The sequences of the genes listed in Table 5 are available in the art and may be obtained from publicly-accessible databases, such as the National Center for Biotechnology Information (http://www.ncbi.nlm.nih.qov/, National Center for Biotechnology Information, National Library of Medicine, Building 38A, Bethesda, Md. 20894), and the European Molecular Biology Laboratory (EMBL) (www.ebi.ac.uk/embl/, EMBL Nucleotide Sequence Submissions, European Bioinformatics Institute, Wellcome Trust Genome Campus, Hinxton, Cambridge, CB10 1SD, UK).

The invention is further illustrated by the following non-limiting examples.

### Materials and Methods Used in the Examples

### GC Cell Lines

GC cell lines were obtained either from commercial sources or collaborators and cultured as recommended. AGS, KatoIII, SNU1, SNU5, SNU16, SNU719, NCI-N87, and Hs746T were obtained from the American Type Culture Collection (http://www.atcc.org/) and cultured as recommended by the supplier. AZ521, Fu97, IM95, Ist1, MKN1, MKN45, MKN7, NUGC3, NUGC4, OCUM1, RerfGC1B Takigawa, TMK1 cells were obtained from the Japanese Collection of Research Bioresources/Japan Health Science Research Resource Bank (http://cellbank.nibio.go.jp/) and cultured as recommended. SCH cells were a gift from Yoshiaki Ito (Institute of Molecular and Cell Biology, Singapore) and grown in RPMI media. YCC1, YCC2, YCC3, YCC6, YCC7, YCC9, YCC10, YCC11, YCC16, YCC17, YCC18, YCC19, and YCC20 cells were a gift from Sun-Young Rha (Yonsei Cancer Center, South Korea) and were grown in MEM supplemented with 10% fetal bovine serum (FBS), 100 units/mL penicillin, 100 units/mL streptomycin, and 2 mmol/L L-glutamine (Invitrogen). CLS145 and HGC27 were obtained from the RIKEN Gene Bank (http://www.brc.riken.go.jp/) and cultured as recommended by supplier.

### Patient Cohorts and Clinical Characteristics

Four independent patient cohorts were analyzed (n=521). Cohort 1 (SG)-200 patients, National Cancer Centre Singapore, Singapore; Cohort 2 (AU)—70 patients, Peter MacCallum Cancer Centre, Australia; Cohort 3 (YG)—65 patients, Yonsei University, South Korea; and Cohort 4 (TMA)—186 patients, National Healthcare Group, Singapore. Cohorts 1-3 (SG/AU/YG) comprise gene expression profiles of primary GCs, while cohort 4 (TMA) comprises tumor sections on a tissue microarray. From the participating centres' tissue repositories or pathology archives, all available primary gastric tumors were collected with approvals from the respective institutional Research Ethics Review Committees and with signed patient informed consent. There was no pre-specified sample size calculation since this is a hypothesis generating discovery study. Clinical information was collected with Institutional Review Board approval and in accordance with REMARK guidelines (McShane L. M. et al., J Natl Cancer Inst, 2005, 97:1180-4). The clinical characteristics of the four cohorts are presented in Table 1. Clinical information was available for all patients except 3 patients in the SG cohort.

### Gene Expression Profiling (GC Cell Lines and Primary Tumors)

For gastric cancer cell lines and patient cohorts 1 and 2, gene expression profiling was performed with Affymetrix Human Genome U133 plus Genechips (HG-U133 Plus 2.0, Affymetrix). For patient cohort 3, IIlumina Human-6 v2 Expression Beadchips was employed. For gastric cancer cell lines and patient cohorts 1 and 2, total RNA was extracted using Qiagen RNA extraction reagents (Qiagen), and hybridized to Affymetrix Human Genome U133 plus Genechips (HG-U133 Plus 2.0, Affymetrix). Raw Affymetrix datasets are available from Gene Expression Omnibus database (GSE15460). For patient cohort 3, total RNA was extracted from the fresh frozen tissues using a mirVana™ RNA Isolation labeling kit (Ambion, Inc.) and hybridized to Illumina Human-6 v2 Expression Beadchips. Primary microarray data is available in the GEO database (GSE 15460 and GSE13861).

### In Vitro Cell Proliferation Assay

Cell proliferation assays were performed using a tetrazolium compound-based colorimetric method. Adherent or semi-adherent cell lines with doubling times less than 48 hours were used in this analysis. The cell lines for which cell proliferation assays were performed are: YCC19, YCC18, TMK1, YCC2, CLS145, YCC9, YCC6, NUGC3, HGC-27, Fu97, Ist1, YCC7, YCC16, Hs746T, MKN45, KatoIII, AGS, SNU719, AZ521, YCC1, MKN1, YCC11, IM95, MKN7, YCC3, YCC10, SCH and N87. Cell proliferation assays were performed using a tetrazolium compound-based colorimetric method (MTS kit, Promega, Madison, Wis., USA) according to the manufacturer's instructions and measured using an EnVision 2104 multilabel plate reader (Perkin Elmer, Finland) at 490 nm. Inhibition of cell growth by drugs was also visually confirmed under microscopy. Drugs used include cisplatin (Sigma, 479306-1G), oxaliplatin (Sigma, O9512), 5-Fluorouracil (Sigma, F6627-1G).

### Histology and Immunohistochemistry

Samples from cohort 1 were subjected to central pathologic review by two independent pathologists (LKH, WWK) blinded to the genomic classification. Immunohistochemical studies using LGALS4 and CDH17 antibodies were performed on a tissue microarray of 186 GC patients (cohort 4), and staining intensities determined by a pathologist blinded to the clinical data (MST). Photomicrographs, details of staining patterns and grading scales are provided below.

### Bioinformatics and Statistical Analysis

Bioinformatic analyses were performed using R. Raw Affymetrix datasets were preprocessed with quantile normalization using RMA (package Affy). Gastric cancer cell lines were filtered using the nsFilter function from the Genefilter package on Bioconductor (Irizarry R. A. et al., Stat Appl Genet Mol Biol, 2003, 2:Article1, hereby incorporated by reference). The R package LIMMA was used for feature selection. Enrichment of functional annotations in the gene expression data were performed using EASE software (http://apps1.niaid.nih.qov/david/; Hosack D. A. et al., Genome Biol, 2003, 4:R70, hereby incorporated by reference). Statistical significance was determined using the Fisher exact score and EASE score. For patient cohorts, preprocessing of cohort 1 and 2 (Affymetrix) was performed with Refplus while preprocessing of cohort 3 (IIlumina) was performed with quantile normalization and the average signal intensity used for summarization. Nearest Template Prediction (Hoshida Y. et al., N Engl J Med, 2008, 359:1995-2004; Reiner A. et al., Bioinformatics, 2003, 19:368-75; Hoshida Y., PLoS One, 2010, 5:e15543, all of which are hereby incorporated by reference) was performed using Genepattern (Reich M. et al., Nat Genet, 2006, 38:500-1, hereby incorporated by reference). The R package e1071 was used for support vector machine (SVM) learning and classification. Correlation with clinico-pathologic parameters and survival analysis were performed using SPSS software (version 16, Chicago). Survival curves were estimated using the Kaplan-Meier method and the duration of survival was measured from the date of surgery to date of death or last follow-up visit. Cancer-specific survival (CSS) was used as the outcome metric, with deaths due to cancer was regarded as an event. Patients who are still alive, died from other causes or lost to follow-up at time of analysis were censored at their last date of follow up. Univariable and multivariable survival analyses were performed using the Cox proportional hazards regression model (Cox D. R., J Royal Stat Soc B, 1972, 34:182-220; Simon R., Br J Clin Pharmacol, 1982, 14:473-82, each of which is hereby incorporated by reference). The test of interaction between the genomic subtypes and therapy was performed with the null hypothesis of treatment equivalence within the subtypes and the alternative hypothesis was of differential treatment efficacy in the subtypes (Cox D. R., J Royal Stat Soc B, 1972, 34:182-220; Simon R., Br J Clin Pharmacol, 1982, 14:473-82, each of which is hereby incorporated by reference). Two-sided p-values less than 0.05 were considered statistically significant. Further details of bioinformatics and statistical analysis are provided below.

### Silhouette Plot Analysis

The Silhouette technique (Rousseeuw P. J., J Comput Appl Math, 1987, 20:53-65, hereby incorporated by reference) was used to evaluate the validity of clustering. To construct the silhouettes S(i) the following formula was used: S(i)=(b(i)−a(i))/max{a(i),b(i)}, where a(i)—average dissimilarity of i-object to all other objects in the same cluster; b(i)—minimum of average dissimilarity of i-object to all objects in other cluster (in the closest cluster). Silhouette values above 0 indicate that the sample is assigned to the appropriate cluster.

### Feature Selection for Intrinsic Signature

Naturally emergent patterns of at least 2 major subtypes within the 37 GCCLs from unsupervised clustering techniques were observed. nsFilter was employed as an initial filter. Briefly, nsFilter removes control probe sets and probe sets without an Entrez Gene ID annotation. A duplicate filter was also used to select the probe set with the largest variance, under conditions where multiple probe sets map to the same Gene ID. Genes were then filtered on variance alone, removing genes with an interquartile range less than the median interquartile range. 10135 genes passed this filter. Hierarchical clustering was performed using Euclidean distance and a complete linkage metric. Using the 2 major subtypes as class labels, LIMMA analysis was performed to identify genes exhibiting differential regulation between the phenotypes2. All signatures were corrected for multiple comparisons by the Benjamini and Hochberg method3 at a q-value threshold of 0.002. These 171 genes constitute the Gastric cell line derived signature associated with the biological subtype distinction.

### Nearest Template Prediction

Prediction analysis was performed by evaluating the expression status of the signature using the nearest template prediction (NTP) method as implemented in the NearestTemplatePrediction module of the GenePattern analysis toolkit. Briefly, a hypothetical sample serving as the template of G-INT outcome was defined as a vector having the same length as the G-INT signature. In this template, a value of 1 was assigned to G-INT-correlated genes and a value of −1 was assigned to G-DIF-correlated genes, and then each gene was weighted by the absolute value of the corresponding t score from the LIMMA algorithm. The template of G-DIF outcome was similarly defined. For each sample, a prediction was made based on the proximity measured by the cosine distance to either of the two templates. Significance for the proximity was estimated by comparison to a null distribution generated by randomly picking (1,000 times) the same number of marker genes from the microarray data for each sample, and correcting for multiple hypothesis testing.

### Support Vector Machine Classifier

A classifier was developed in the training gastric cancer cell line dataset based upon class labels generated by unsupervised hierarchal clustering of gastric cancer cell lines. A Support-Vector Machine (SVM) classification algorithm with a Radial-Basis Function (RBF) Kernel and eps-regression option was used, as provided by the Bioconductor software package e1071. After cross-validation, the trained classifier was then applied to the target primary tumor datasets. Each tumor profile is then ascribed a predicted class label, based on their classification scores (scaled SVM scores) reflecting the similarity of that sample with either G-INT or G-DIF subclass respectively.

### Concordance Between Both Classification Systems

Concordance between the 2 classification systems was 91-94% for the training dataset (GC cell lines) as well as in primary tumors (SG and AU cohorts). 86% of samples were identified by NTP at an FDR of <0.05. These results show that the 171 gene set can robustly classify primary tumors into G-INT and G-DIF sub-classes.

### Tissue Microarrays

A total of 186 gastric cancer cases that were surgically resected at the National University of Singapore between year 2000 and 2008 were included in the construction of the tissue microarray (TMA). The TMA blocks were constructed as described previously (Zhang D. et al., Mod Pathol, 2003, 16:79-84; Ong C. W. et al., Mod Pathol, 2010, 23:450-7, each of which is hereby incorporated by reference). Briefly, a needle with 0.6 mm diameter was used to punch a donor core from morphologically representative areas of a donor tissue block. The core was subsequently inserted into a recipient paraffin block using an ATA-100 tissue arrayer (Chemicon, USA). Each core was taken from the central of tumor growth as well as a separate core from the matched histologically-normal gastric epithelium of the same case. Consecutive TMA sections of 4 μm thickness were cut and placed on slides for immunohistochemical analyses.

### Immunohistochemical Procedures

All protein markers were assessed immunohistochemically using commercially available antibodies (see table below). Antigen retrieval was carried out with 10 mM citrate buffer (pH 6.0) in a MicroMED TT Microwave Processor (Milestone, Sorisole, Italy) for 5 minutes at 120° C. Slides were then incubated with the primary antibody for 12 hours at the dilutions indicated in the table below. Immunostaining was performed with the streptavidin-biotin kit (LSAB2, Dako, Norway) in accordance with the manufacturer's specifications and the slides were then counterstained with hematoxylin. Various human tissues or cell lines embedded in paraffin with known expression for the markers were used as positive controls. Paraffin-embedded colorectal cancer tissue specimens were used as positive control for CDH17 (Su M. C. et al., Mod Pathol, 2008, 21:1379-86, hereby incorporated by reference). For LGALS4, normal colonic epithelial tissues were used as positive controls (Huflejt M. E. et al., Glycoconj J, 2004, 20:247-55, hereby incorporated by reference). Negative controls consisted of the omission of primary antibody without any other changes to subsequent procedures.

### Dilutions Used and Manufacturers Information for Antibodies Used in the Immuno-Histochemical Assays:

| G-INT | | | |
|---|---|---|---|
| Marker | Dilution | Clone | Manufacturer |
| CDH17 | 1:1000 | 1E8 | Sigma-Aldrich, MO, USA |
| LGALS4 | 1:200 | 1H3 | Sigma-Aldrich, MO, USA |

### Scoring for Protein Expression

Dark brown membranous staining was defined as positive for CDH17. Positivity of LGALS4 was defined as staining in the cytoplasmic compartment. The staining was scored as follows: 0 (no detectable staining); 1+ (<25% positive cells), 2+ (25-49%) and 3+ (>50%). The primary evaluation of the staining was independently performed by a trained scientist (CWO) and confirmed by a gastrointestinal pathologist (MST).

### Statistical Test for Interaction

The test of interaction between the intrinsic genomic subtypes and therapy were performed with the null hypothesis of treatment equivalence within the subtypes, and the alternative hypothesis of differential treatment efficacy between the subtypes (Cox D. R., J Royal Stat Soc B, 1972, 34:182-220; Simon R., Br J Clin Pharmacol, 1982, 14:473-82, each of which is hereby incorporated by reference). For the test of interaction (null hypothesis=NO interaction between therapy and genomic subtypes; alternative hypothesis=interaction between therapy and genomic subtypes), the model takes the form:

λgt(τ)=f(τ)exp(ag+bt+cgt);

with the hypotheses defined as:

H0: cg=1; t=1=cg=1; t=2=cg=2; t=1=cg=2; t=2=0 and

HA: At least 1 interaction term is not zero (cg=i; t=j≠0)

If the null hypothesis is rejected, subset effects will be investigated and the model above will be abandoned. The subset HR will be calculated based on 4 different models. Taking g=1 to define Subtype 1, g=2 to define Subtype 2, t=1 to define Adjuvant 5-FU based treatment and t=2 to define Surgery alone, the 4 models are as follows:
1. λgt(τ)=f(τ)exp(ag); Analysis done only on subset: patients on Adjuvant 5-FU based treatment
2. λgt(τ)=f(τ)exp(ag); Analysis done only on subset: patients on Surgery alone
3. λgt(τ)=f(τ)exp(bt); Analysis done only on subset: patients with Genomic Subtype 1
4. λgt(τ)=f(τ)exp(bt); Analysis done only on subset: patients with Genomic Subtype 2
Effectively model 1 and 2 are the same only that the patients used for the analysis are two different groups (mutually exclusive groups). The same goes for Model 3 and 4. An example is provided in Table 4.

### Example 1

### Genomic Analysis of GC Cell Lines Reveals Two Major Intrinsic Subclasses

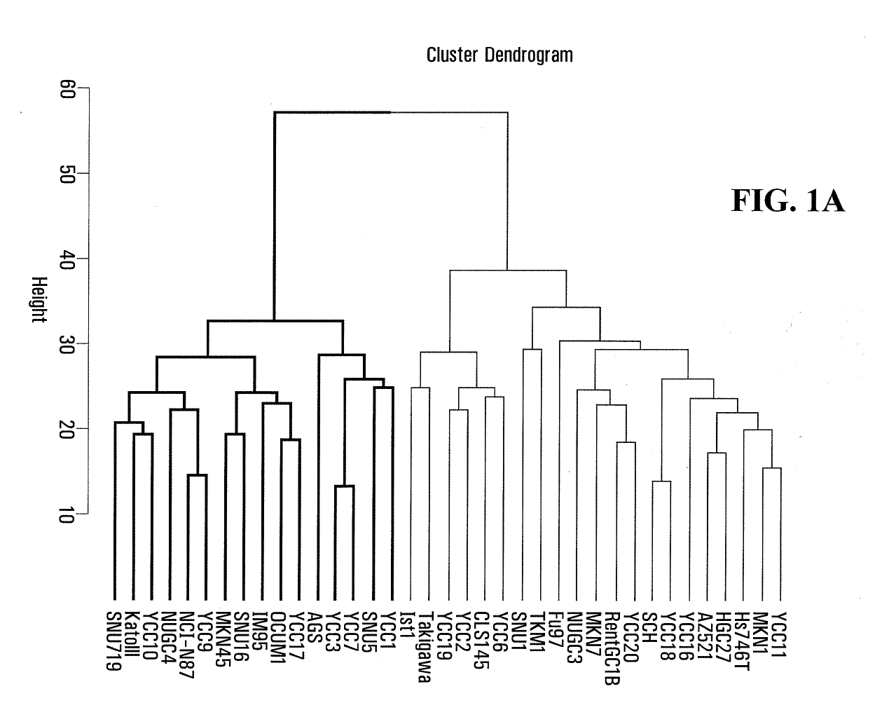

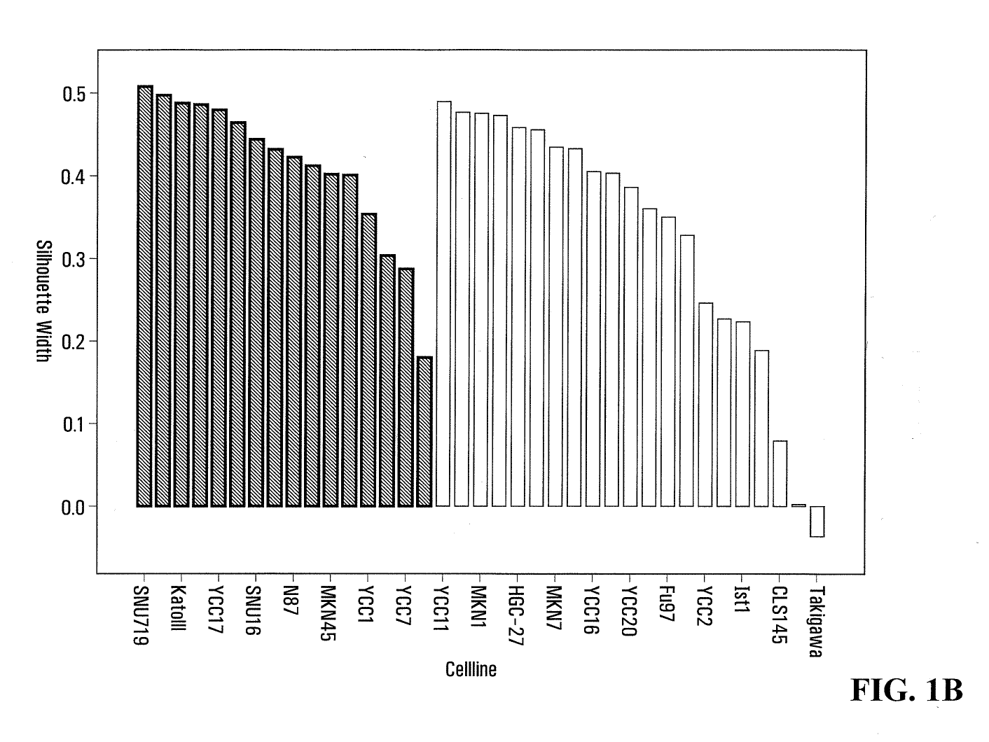

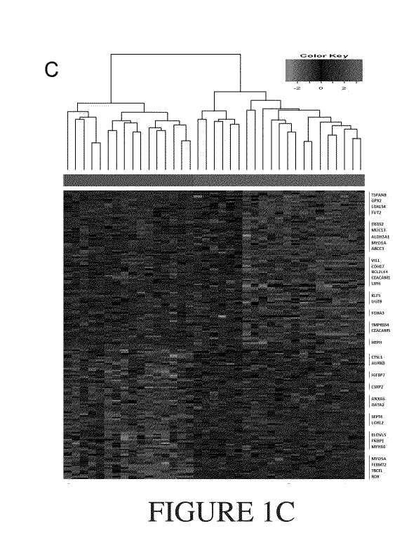

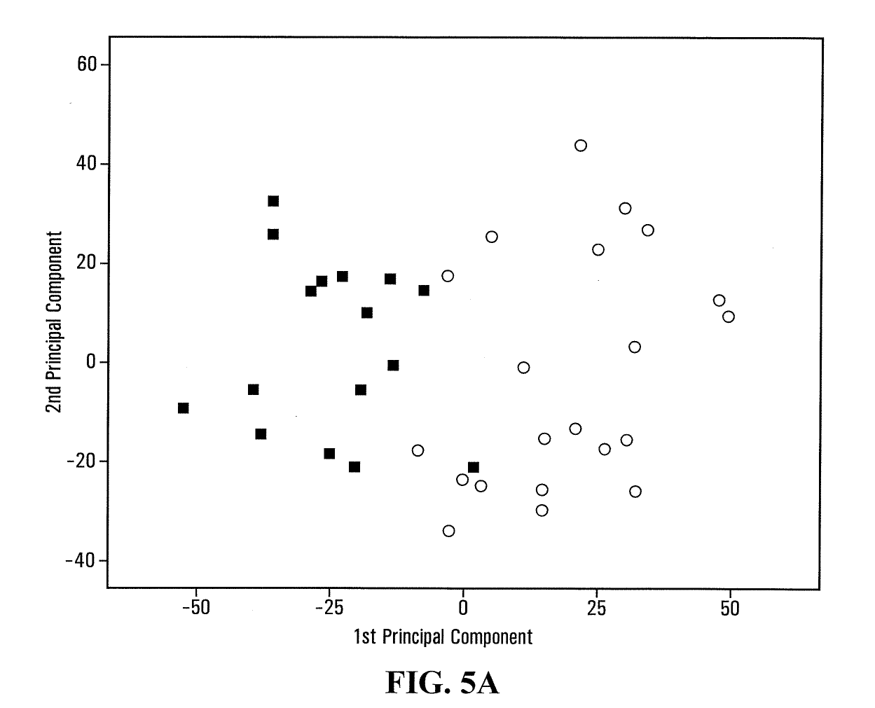

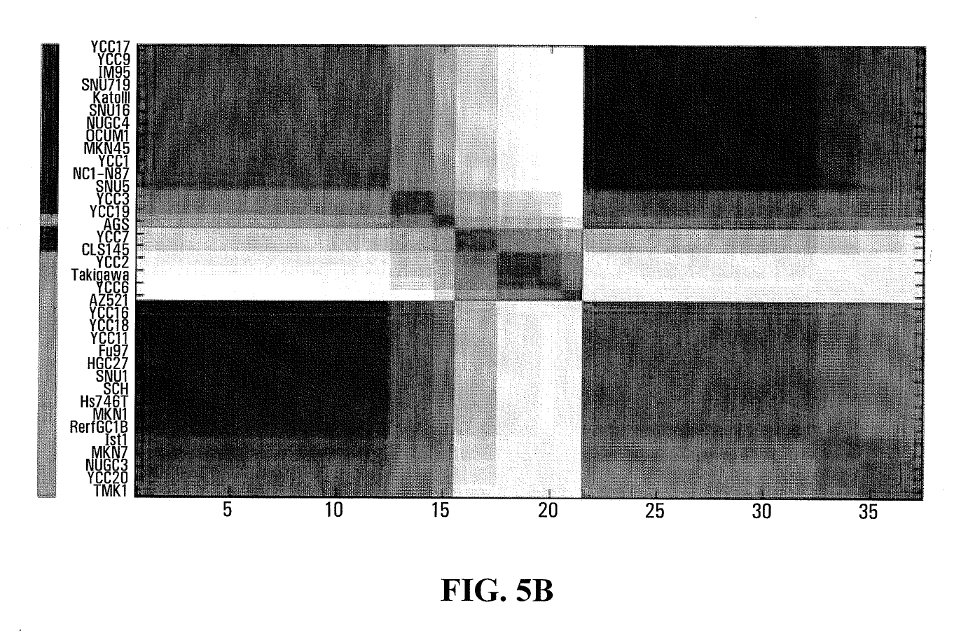

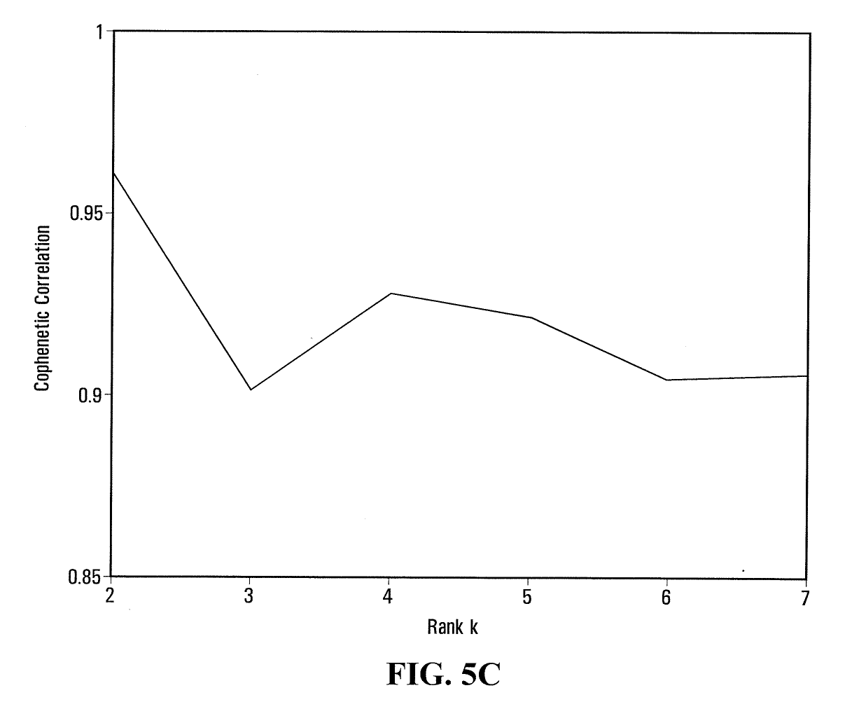

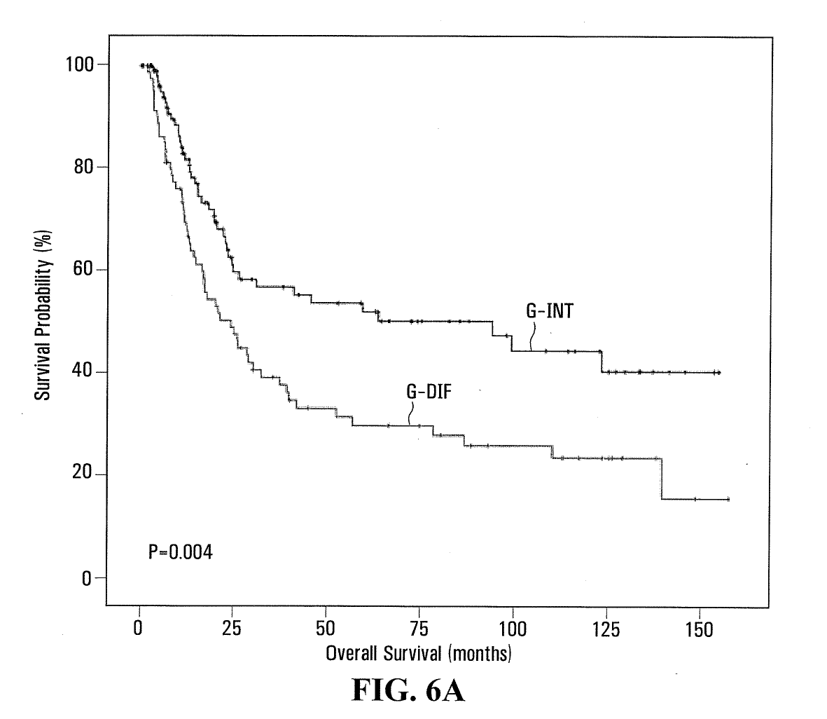

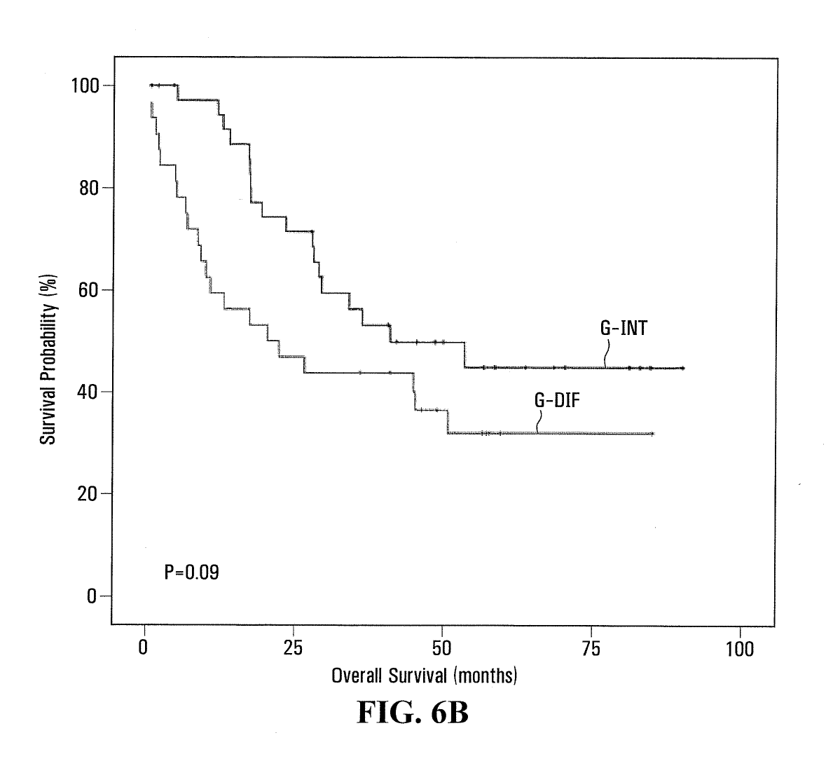

Gene expression profiling was performed for a panel of 37 GC cell lines. Analysis of the expression data using four different unsupervised and unbiased clustering techniques (hierarchical clustering (Eisen M. B. et al., Proc Natl Acad Sci USA, 1998, 95:14863-8, hereby incorporated by reference), silhouette plot (SP) analysis (Rousseeuw P. J., J Comput Appl Math, 1987, 20:53-65, hereby incorporated by reference), nonnegative matrix factorization (NMF) (Lee D. D. et al., Nature, 1999, 401:788-91, hereby incorporated by reference), and principal components analysis (PCA)) was performed to identify pervasive and thereby “intrinsic” gene expression differences across the cell lines. Two major intrinsic subtypes were identified by hierarchical clustering (FIG. 1A). The robustness of the subtypes was further verified by SP, NMF, and PCA analysis (FIG. 1B and FIG. 5). These two intrinsic subtypes are henceforth referred to as Genomic intestinal (G-INT) and Genomic Diffuse (G-DIF).

### Example 2

### The Intrinsic Subtypes are Associated with Highly Distinctive Gene Expression Patterns

LIMMA (Linear models for microarray data) (Smyth G. K., Stat Applications Gen Mol Biol, 2004, 3:Article 3, hereby incorporated by reference), a modified t-test incorporating the Benjamini Hochberg multiple correction technique (Benjamini Y. et al., Behav Brain Res, 2001, 125:279-84, hereby incorporated by reference), was used to analyze gene expression differences between the intrinsic subtypes. A genomic signature of 171 genes was identified, distinguishing the G-INT and G-DIF intrinsic subtypes (FDR<0.002; FIG. 1C and Table 5). A search was performed for potentially redundant features among the 171 gene set. Comparing the correlation coefficients of the 171 genes to one another showed that only 2 of the 171 genes exceeded a pre-defined correlation threshold of 0.88. Given this lack of redundancy, further analysis was performed using the entire 171 gene set. Expression Analysis Systematic Explorer (EASE) [27] was applied to the genomic signature to identify biological themes within the genes up-regulated in either subtype (http://david.abcc.ncifcrf.gov/ease/ease.jsp). Genes up-regulated in the G-INT subtype were enriched for functions related to carbohydrate and protein metabolism (FUT2) and cell adhesion (LGALS4, CDH17) (within system FDR<0.01), while cell proliferation (AURKB) and fatty acid metabolism (ELOVL5) functional annotations (within system FDR<0.01) were enriched within genes up-regulated in the G-DIF subclass (Table 6). The two intrinsic subtypes, GINT and G-DIF, are thus associated with highly distinctive gene expression patterns and biological pathways.

### Example 3

### The Intrinsic Subtypes are Recurrently Observed in Primary Tumors

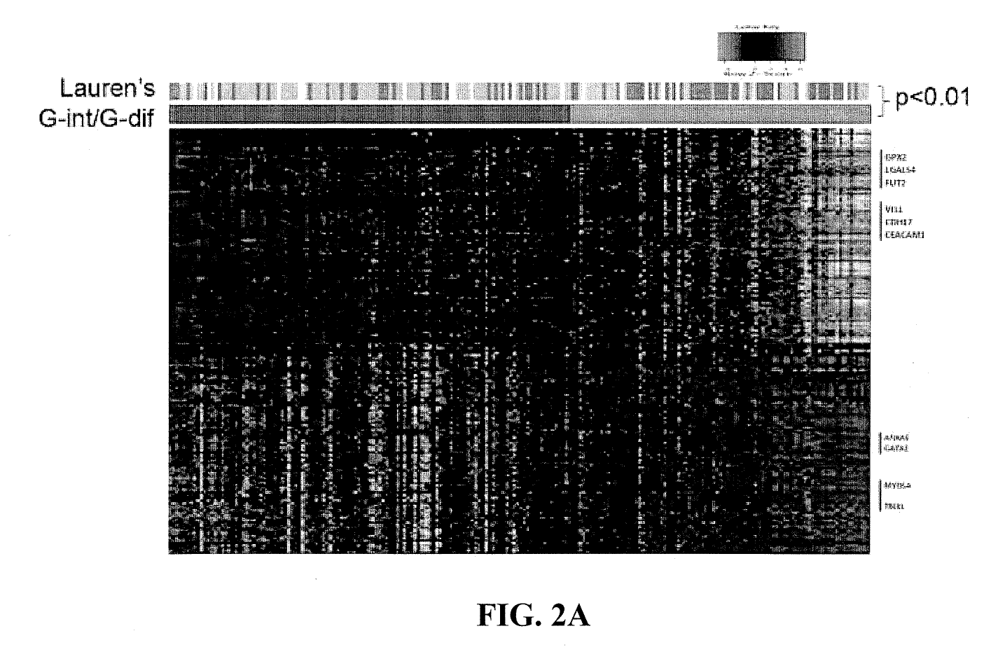

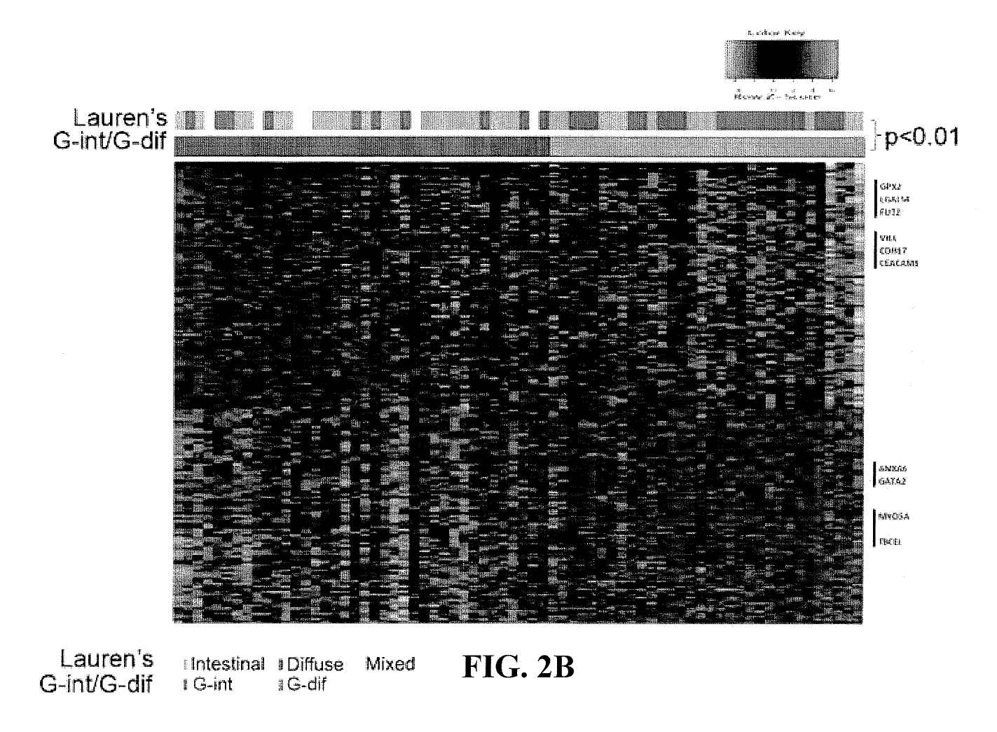

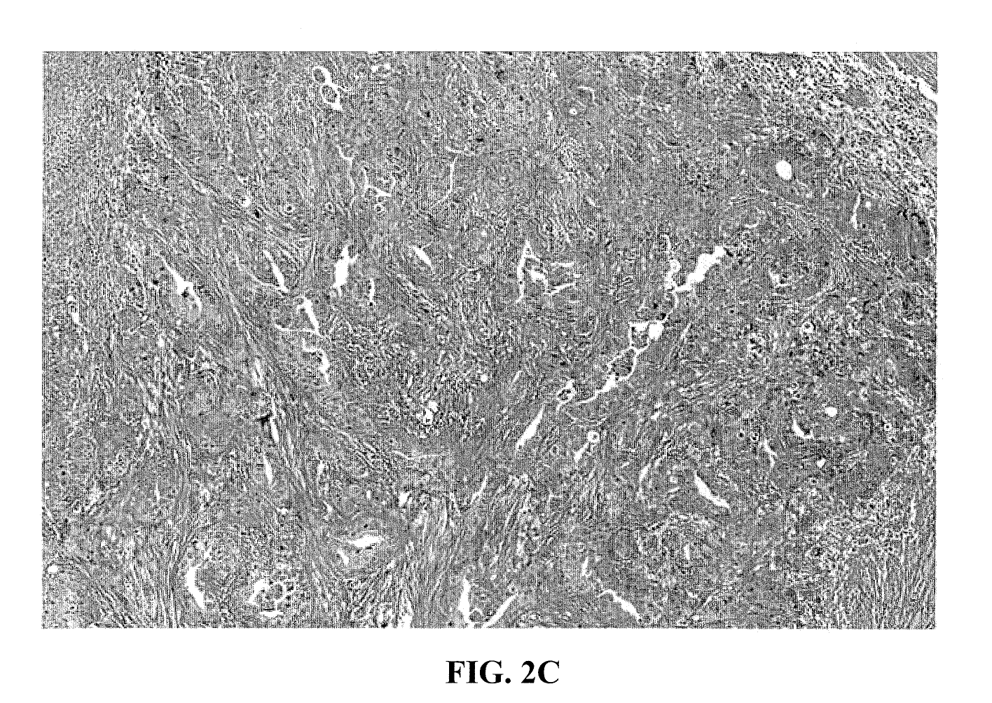

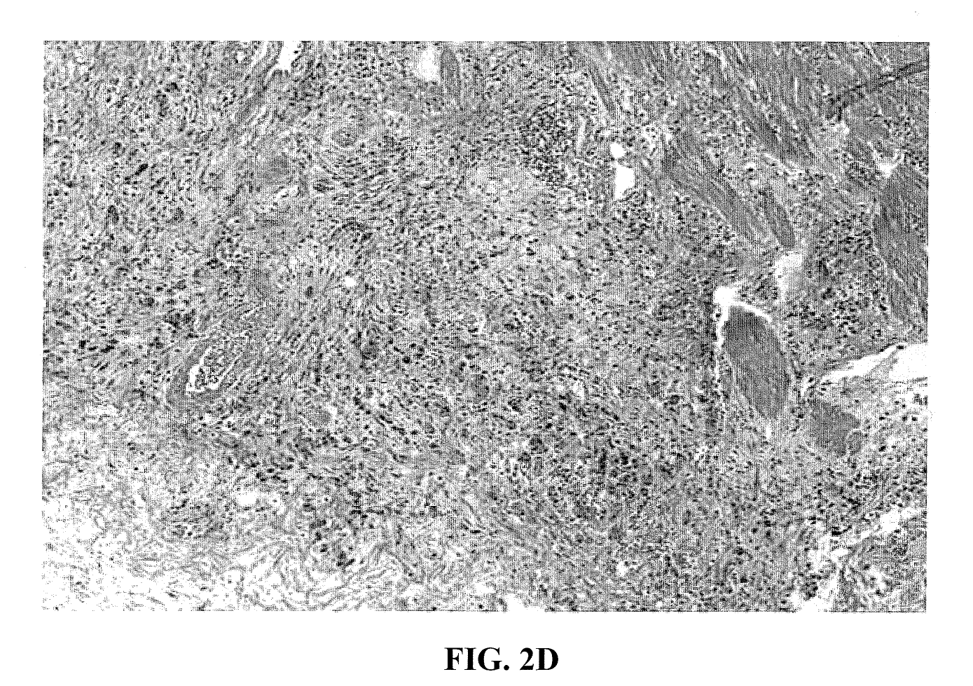

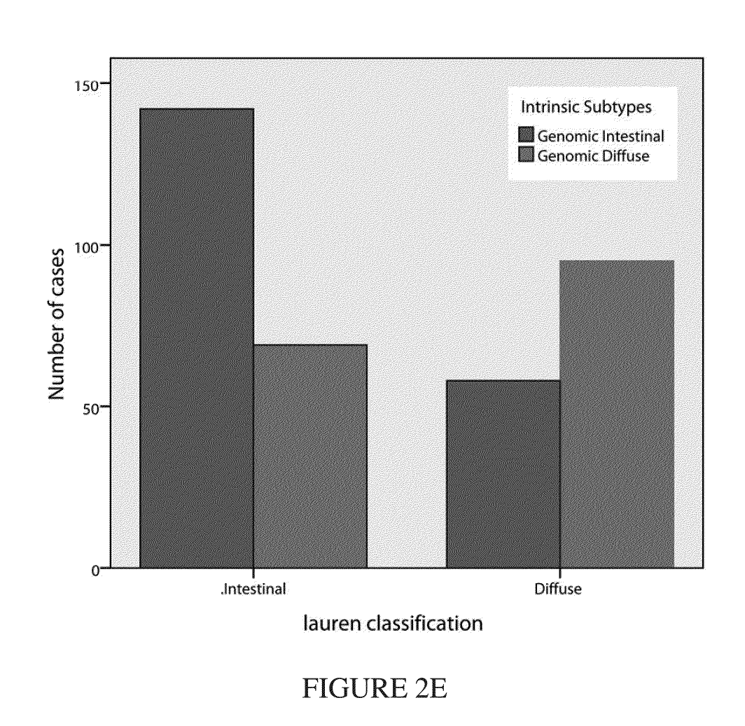

The intrinsic 171-gene genomic signature was mapped onto primary tumors in two independent cohorts of GC patients (SG and AU), collectively totaling 270 patients. Two classification algorithms were used (Nearest Template Prediction and a support vector machine classifier). Concordance between the 2 classification systems (SVM and NTP) was 94-96% in the SG and AU cohorts with 88% of samples identified by NTP at an FDR of <0.05. These results show the 171 gene set can robustly classify primary tumors into G-INT and G-DIF sub-classes. Due to its methodological simplicity and applicability to single samples without requiring a corresponding training dataset [30], the NTP classifications were used for subsequent analyses. Specifically, 114 samples in the SG cohort and 38 samples in the AU cohort were classified as G-INT (FIGS. 2 A & B and Table 7).

### Example 4

### The Intrinsic Subtypes are Partially Associated with Lauren's Histopathologic Classification

The associations of the intrinsic subtypes with clinical-pathologic parameters was investigated. The intrinsic subtypes were found to be significantly associated with Lauren's intestinal and diffuse subtypes respectively in the SG (p=0.002) and AU cohorts (p=0.003), hence their name (G-INT and G-DIF). Besides Lauren's, the intrinsic subtypes were also related to tumor grade (Table 7).

Although the intrinsic subtypes are named G-INT and G-DIF due to their associations with Lauren's histopathology, the overall concordance between the intrinsic genomic subtypes and Lauren's histopathology was only 64%. Thus, the two classifications should more appropriately be regarded as related but distinct. Specifically, 91 of 134 Lauren's intestinal cases were classified at GINT, and 64 of 106 Lauren's diffuse cases were classified as G-DIF (FIGS. 2 A & B). These discrepancies are unlikely to be due to inter-pathologist differences alone, as pathologic review in the SG cohort had been performed by 2 independent pathologists blinded to the genomic classification (Representative H & E slides of discordant tumors are also presented in FIGS. 2 C & D). Rather, the intrinsic genomic signature may capture salient features of the tumor that are less obvious to discern by light microscopy.

### Example 5

### The Intrinsic Subtypes are Independently Prognostic of Patient Survival

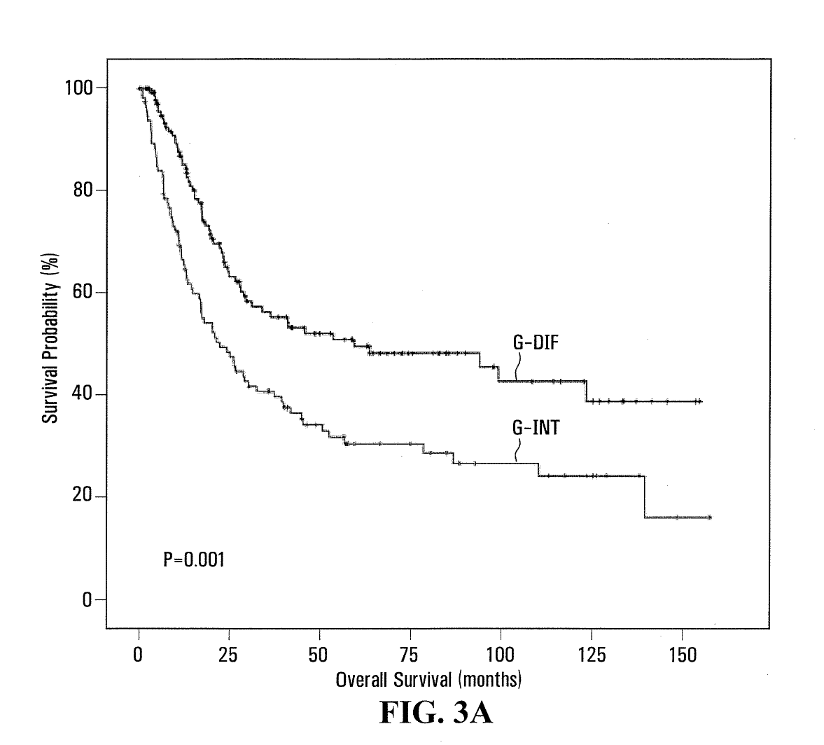

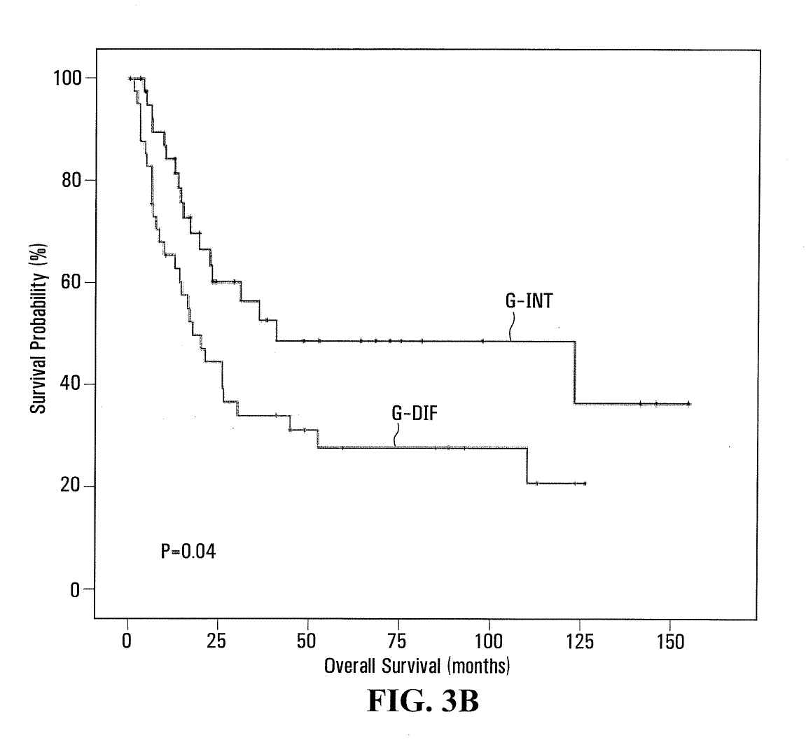

Using cancer-specific survival as the outcome metric, patients with G-DIF cancers had worse survival outcomes compared to patients with G-INT tumors in the SG and AU cohorts (cohort 1: HR 1.78, 95% Cl: 1.19-2.64, p=0.004; cohort 2: HR 1.73, 95% Cl: 0.92-3.26, p=0.09) and also in a combined analysis (HR: 1.79, 95% Cl: 1.28-2.51, p=0.001, FIG. 3A). In contrast, Lauren's classification was not prognostic (p=0.23). Further supporting the prognostic relevance of the intrinsic subtypes, in discordant cases, patients with G-INT but diffuse type cancers exhibited superior survival compared to patients with G-DIF but intestinal type cancers (HR 1.83, 95% Cl: 1.02-3.30, p=0.04, FIG. 3B).

In a multivariate analysis (Table 2), the intrinsic subtypes remained prognostic (p<0.001) even after accounting for other interacting factors such as Lauren's classes and grade. The intrinsic subtypes were also prognostic after accounting for other variables that were also prognostic in univariate analysis (stage, margin status and gender; p=0.005).

### Example 6

### The Intrinsic Subtypes are Prognostic in an Independent Patient Cohort Profiled by a Different Microarray Platform

To further determine the general applicability of the intrinsic subclasses, the intrinsic genomic signature was applied to a third GC patient cohort (YG) profiled on a different microarray platform (Illumina Human-6 v2 Expression Beadchip). Of the 65 patients, 35 were classified as G-INT by NTP. Similar to the SG and AU cohorts, patients with G-INT tumors had superior overall survival compared to patients with G-DIF tumors in the YG cohort (HR 3.3, 95% Cl: 1.03-10.53, p=0.04), while Lauren's classes was not prognostic (p=0.23).

### Example 7

### G-INT Patients Identified by Immunohistochemical Markers Exhibit Improved Survival Outcomes

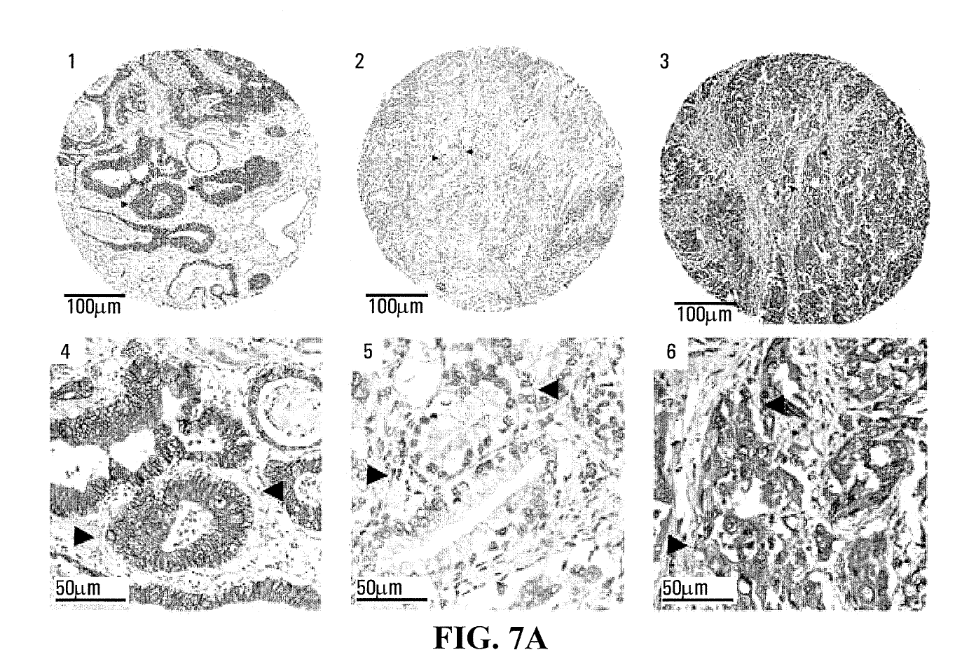

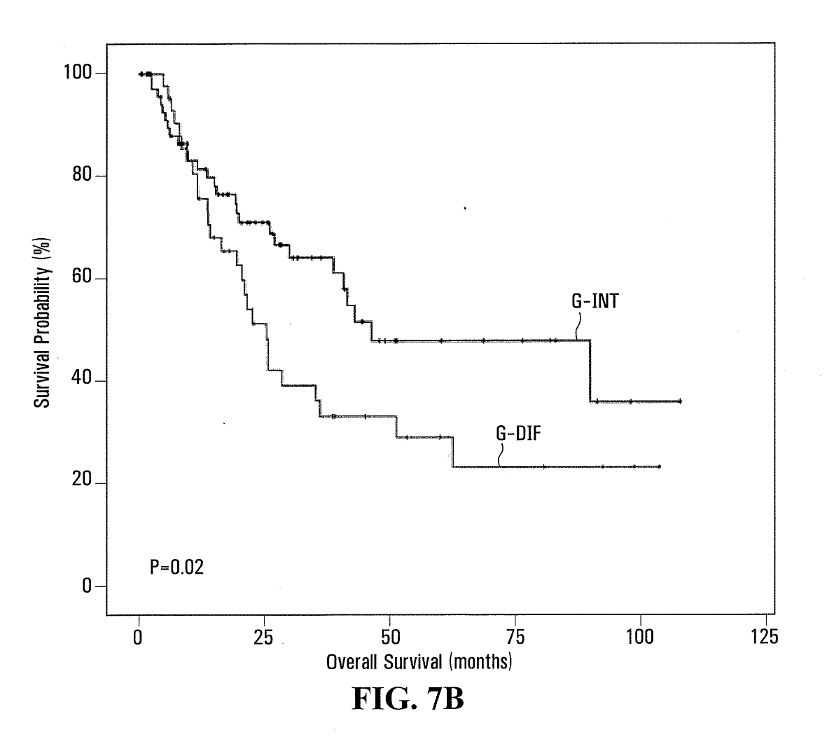

To assess if a panel of immunohistochemical markers might also be used to identify the intrinsic subtypes and its relation to survival outcomes, an independent tissue microarray (TMA) cohort (cohort 4) of 186 GC patients was analyzed. Two G-INT markers were selected (LGALS4 and CDH17) meeting the criteria of high gene expression in G-INT cell lines and tumors, and for which commercial immunohistochemical markers were available. The TMA tumors were classified based on their intensity of LGALS4 and CDH17 staining (CDH17 (>1+) and LGALS4 (>2+)), using intensity cutoffs determined by a pathologist blinded to the clinical data. To confidently distinguish between G-INT and G-DIF cancers, the 2-marker positive group (G-INT) was compared to the 2-marker negative group (G-DIF). Among the 186 tumors, 75 were classified as G-INT (both markers positive), 44 as G-DIF (neither marker positive) and 67 were equivocal (one marker positive). Patients with G-DIF tumors classified by IHC exhibited worse outcomes than G-INT tumors classified by IHC (Hazard ratio, adjusted for stage: 1.95, 95% Cl: 1.13-3.38, p=0.02) (FIGS. 7A & B), while Lauren was once again not prognostic (p=0.33).

### Example 8

### The Intrinsic Subtypes Exhibit Distinct In Vitro Responses to Chemotherapy

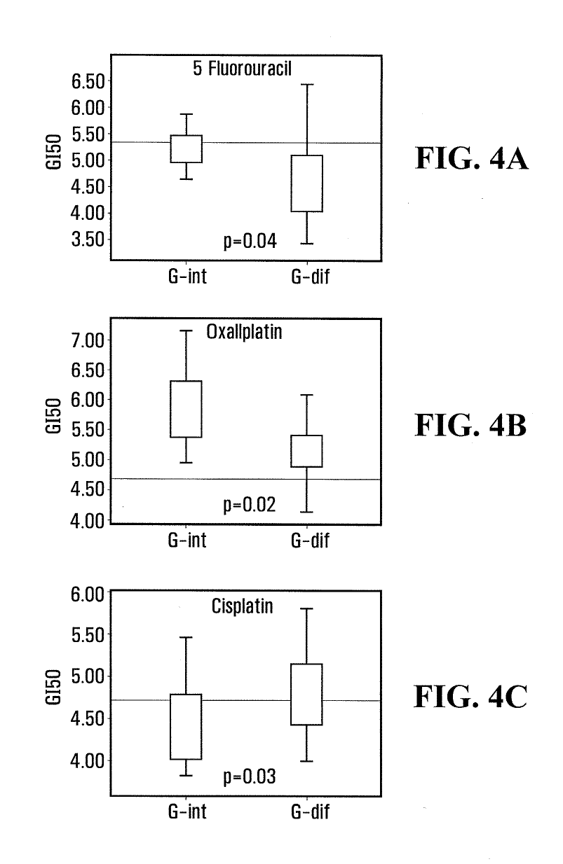

Of the 37 cell lines, 28 cell lines (11 G-INT and 17 G-DIF) had growth characteristics suitable for in vitro drug sensitivity testing. 5-FU, oxaliplatin and cisplatin are drugs presently employed in the adjuvant and 1st line palliative treatment of GC. The 28 cell lines were treated with increasing concentrations of these drugs. G-INT cell lines were significantly more sensitive to 5-FU (p=0.04) and oxaliplatin (p=0.02) in vitro, while G-DIF cell lines were more sensitive to cisplatin (p=0.03) (FIG. 4, see legend for mean drug concentrations). The in vitro dosages used are comparable to therapeutic ranges observed in human patients based on pharmacokinetic analysis (Saif M. W. et al., J Natl Cancer Inst, 2009, 101:1543-52; Ikeda K. et al., Jpn J Clin Oncol, 1998, 28:168-75; Graham M. A. et al., Clin Cancer Res, 2000, 6:1205-18, all of which are hereby incorporated by reference) (FIG. 4). These results point to differential in vitro sensitivities of G-INT cell lines to 5-FU and oxaliplatin, and G-DIF cell lines to cisplatin.

### Example 9

### G-INT Patients may Derive Differential Benefit from 5-FU Treatment

Information regarding use of adjuvant 5 Fluorouracil chemoradiation were available from 2 gene expression cohorts (1 & 2) and the TMA cohort (cohort 4). Decisions regarding adjuvant therapy in these cohorts were based upon existing knowledge at the point of diagnosis, patient's general health status, risk factors for relapse especially disease stage, treatment related toxicities and patient preference.

Patients with advanced stage disease were more likely to receive adjuvant treatment (p=0.03), however no significant differences were observed in prescribing 5-FU therapy between the intrinsic subtypes either across all stages (p=0.27) or within each stage (p˜0.4-0.8) (Table 7). To evaluate if the intrinsic subtypes might exhibit differential benefit with 5-FU chemoradiation in the patient cohorts, a statistical test for interaction that was specifically adjusted for stage was performed.

A significant interaction between the intrinsic subtypes and benefit with 5-FU based chemoradiation (Table 3) was observed, which shows that patients with G-INT tumors may derive differential benefit from adjuvant 5-FU based therapy. Specifically, the test for interaction by Cox proportional hazards regression was p=0.002 (combined analysis), gene expression (p=0.03) and TMA cohorts (p=0.02). The stage adjusted hazard ratio of death due to cancer for surgery alone compared to adjuvant 5-FU therapy was 1.68 (p=0.06 for G-INT tumors and 0.90 (p=0.67) for G-DIF tumors. Table 3 presents the interactions for the combined analysis, while the gene expression and TMA cohorts are separately presented in Table 8.

### Example 10

### Bioinformatic Analysis

1. Naturally emergent patterns of at least 2 major subtypes within gene expression profiles from 37 Gastric Cancer Cell Lines (GCCLs) issuing from unsupervised clustering techniques was observed (hierarchal clustering, NMF clustering, Kmeans clustering, silhouette plot analysis).

2. Feature selection. Bioinformatic analysis was performed with R.

a. To select features, nsFilter was employed as an initial filter.

i. Briefly, nsFilter removes control probe sets and probe sets without an Entrez Gene ID annotation. A duplicate filter was also used to select the probe set with the largest variance, under conditions where multiple probe sets map to the same Gene ID. Genes were then filtered on variance alone, removing genes with an interquartile range less than the median interquartile range. 10135 genes passed this filter.

ii. Hierarchical clustering was performed using Euclidean distance and a complete linkage metric.

iii. Using the 2 major subtypes as class labels, LIMMA analysis (package e1071 from bioconductor) was performed to identify genes exhibiting differential regulation between the phenotypes.

iv. All analysis were corrected for multiple comparisons by the Benjamini and Hochberg method3 at a q-value threshold of 0.002.

v. These 171 genes constitute the Gastric cell line derived signature associated with the biological subtype distinction.

3. Classification. Nearest Template Prediction was performed with GenePattern (publicly available at www.broadinstitute.org/cancer/software/genepattern/)

i. Prediction analysis was performed by evaluating the expression status of the signature using the nearest template prediction (NTP) method as implemented in the NearestTemplatePrediction module of the GenePattern analysis toolkit.

ii. Briefly, a hypothetical sample serving as the template of G-INT outcome was defined as a vector having the same length as the GINT signature. In this template, a value of 1 was assigned to G-INTcorrelated genes and a value of −1 was assigned to G-IFcorrelated genes, and then each gene was weighted by the absolute value of the corresponding t score from the LIMMA algorithm. The template of G-DIF outcome was similarly defined.

iii. For each sample, a prediction was made based on the proximity measured by the cosine distance to either of the two templates. Significance for the proximity was estimated by comparison to a null distribution generated by randomly picking (1,000 times) the same number of marker genes from the microarray data for each sample, and correcting for multiple hypothesis testing.

iv. An FDR<0.05 defines a robustly classified sample.

4. How many genes to robustly classify. The table in subsequent pages of this document list all 171 genes ranked from most “discriminative” to least “discriminative”. The subsequent table list effects of dropping genes from the bottom of the list, leaving behind the top 170, top 169 genes and so on. It appears that dropping below 60 genes compromises slightly on the precision of the classification and dropping below 44 substantially on the precision of the classification.

### Example 11

### Comparison of the Classification Precision and Prognostic Performance of an Intrinsic Gastric Cancer Signature with Existing Genomic Signatures in Six Independent Datasets

Background:

Several gene expression signatures derived from supervised approaches based on histology, peritoneal or lymph node metastases and survival have been proposed in order to classify gastric cancers such as adenocarcinomas and provide prognostic information. These studies had relatively small sample sizes. There are two major disadvantages of these approaches. One disadvantage is that gastric adenocarcinomas are characterized by substantial tissue heterogeneity. Different cell populations (tumor cells, fibroblastic/desmoplastic stroma and immune cells) may confound signature development and use thereof. Macro and micro-dissection can be challenging. Another disadvantage is that supervised approaches rely on precise histopathology. Discordance among pathologists compromises signature development. The strategy described in this example involves an initial focus on a diverse panel of gastric cancer cell lines. The hypothesis is that any genomic differences detected in cell lines should be, by nature, tumor-centric and thereby “intrinsic” to the underlying biology of the GC cancer cell.

Methods:

7 datasets of gene expression profiles across different microarray platforms were generated in-house or obtained from collaborators. The study included a panel of 37 gastric cancer cell lines (GCCLs) which were analyzed using the Affymetrix U133-2Plus microarray and samples from 549 patients in 6 independent patient cohorts as follows: 197 patients in Singapore whose samples were analyzed using the Affymetrix U133-2plus microarray; 70 patients in Australia, whose samples where analyzed using the Affymetrix U133-2plus microarray: 31 patients in the United Kingdom whose samples were analyzed using the Affymetrix U133AB microarray; 90 patients from Hong Kong whose samples were analyzed using a custom array; a first set of 96 patients from Korea whose samples were analyzed using a custom array; and a second set of 65 patients in Korea whose samples were analyzed using the Illumina Human-6 v2 microarray. Unsupervised techniques were used to distinguish major intrinsic subtypes from GCCLs and distinguishing features were identified using linear models for microarray data (LIMMA). Patient tumors were classified using the nearest template prediction algorithm and the classification precision and correlation with patient survival were evaluated.

Results:

Beginning with unsupervised techniques, 2 major intrinsic subtypes were identified from the training set (GCCL). A 171-gene signature was identified that could distinguish the two subtypes of tumors. At a false discovery rate of 0.05, the signature precisely classified 432 (78.6%—see Table 11) of primary tumors with 61.1% to 88.6% of tumors precisely classified in each dataset and 55% of the classified tumors belonging to the larger of 2 intrinsic subgroups. With 5 other published signatures, classification precision was <30%. The 2 genomic subtypes were differentially enriched among Lauren's intestinal and diffuse histological subtypes (p<0.001, chi square test). The subclasses were therefore referred to as genomic intestinal and genomic diffuse (FIG. 2E).

This classification of intrinsic subtypes provided prognostic information with the more aggressive subgroup having inferior overall survival: median survival: 30 months vs. 71 months (HR 1.48; 95% Cl: 1.14-1.92, p<0.01, univariate analysis and HR 1.39; 95% Cl: 1.05-1.78, p=0.02 after adjusting for stage—See Table 12). All of the other previously published gene signatures were found to be not prognostic.

The genomic intrinsic gastric cancer classification scheme described herein which was discovered by an unsupervised approach in investigating gastric cancer cell lines precisely classifies patient samples. Although the intrinsic subtypes classification is related to Lauren's histology, it represents a significant improvement by providing independent prognostic value in 6 independent datasets across different microarray platforms.

This example indicates that the intrinsic signature provided by the method described herein was successful in precisely classifying gastric cancers in 6 large patient cohorts from different countries and using different microarray platforms. This indicates that the methods described herein provide better prognostic information than the methods that use the previously existing signatures.

The citation of any publication is for its disclosure prior to the filing date and should not be construed as an admission that the present invention is not entitled to antedate such publication by virtue of prior invention.

Unless defined otherwise, all technical and scientific terms used herein have the same meaning as commonly understood to one of ordinary skill in the art to which this invention belongs. As used in this specification and the appended claims, the singular forms “a,” “an,” and “the” include plural reference unless the context clearly dictates otherwise.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

TABLE 1
Clinical Characteristics of Patient Cohorts. Clinical information is
available for all but 3 patients in the SG cohort. Median follow-up for
patients still alive for the 4 cohorts are 33, 56, 39 and 36 months
respectively.

| | | | |
|---|---|---|---|
| SG | AU | YG | TMA |
| (n = 197) | (n = 70) | (n = 65) | (n = 186) |

Age

| | | | | |
|---|---|---|---|---|
| range | 23-92 | 32-85 | 32-83 | 31-87 |
| mean, S.D | 64.6, 13.1 | 65.5, 12.5 | 61.0, 11.5 | 65.8, 11.7 |

Gender

| | | | | |
|---|---|---|---|---|
| Male | 128 | 48 | 46 | 128 |
| Female | 69 | 22 | 19 | 58 |

Lauren's

| | | | | |
|---|---|---|---|---|
| Intestinal | 100 | 34 | 22 | 97 |
| Diffuse | 76 | 30 | 31 | 46 |
| Mixed | 21 | 6 | 12 | 43 |

Grade

| | | | | |
|---|---|---|---|---|
| Moderate to well | 72 | 24 | 40 | 52 |
| differentiated | | | | |
| Poorly differentiated | 125 | 46 | 25 | 134 |

Stage

| | | | | |
|---|---|---|---|---|
| 1 | 31 | 13 | 12 | 12 |
| 2 | 32 | 16 | 2 | 68 |
| 3 | 72 | 33 | 35 | 57 |
| 4 | 62 | 8 | 16 | 49 |

Adjuvant 5-FU based therapy (in eligible patients)

| | | | | |
|---|---|---|---|---|
| Yes | 36 | 28 | Not available | 19 |
| No | 123 | 31 | | 70 |

Surgical Margins

| | | | | |
|---|---|---|---|---|
| Negative | 169 | 66 | Not available | 162 |
| Positive | 28 | 4 | | 24 |

TABLE 2
Multivariable Cox proportional hazards models. Model (1) incorporates
G-INT/G-DIF classes together with Lauren's classes and histological
grade which were found to be associated with G-INT/G-DIF subtypes.
Patients with mixed histology were excluded from Model (1), Model (2)
incorporates all variables found to be prognostic on univariate analysis.
Statistically significant results are in bold.

| | |
|---|---|
| | Multivariable, |
| Univariate, | HR (95% CI), |
| HR (95% CI), p value | p value |

Model (1): Factors interacting with G-INT/G-DIF subtypes

| | | | |
|---|---|---|---|
| G-INT/ | G-INT | 1.00 | 1.00 |
| G-DIF | | | |
| | G-DIF | 1.95 (1.36-2.78), | 1.92 (1.32-2.78), |
| | | p < 0.001 | p < 0.001 |
| Grade | Moderate/Well | 1.00 | 1.00 |
| | differentiated | | |
| | Poor/ | 1.41 (0.98-2.04), | 1.40 (0.85-2.31), |
| | undifferentiated | p = 0.07 | p = 0.19 |
| Lauren's | Intestinal | 1.00 | 1.00 |
| | Diffuse | 1.24 (0.87-1.76), | 0.81 (0.50-1.32), |
| | | p = 0.23 | p = 0.40 |

Model (2): Factors affecting survival in univariate analysis

| | | | |
|---|---|---|---|
| G-INT/ | G-INT | 1.00 | 1.00 |
| G-DIF | | | |
| | G-DIF | HR: 1.79, (1.28-2.51), | 1.63 (1.16-2.29), |
| | | p = 0.001 | p = 0.005 |
| Gender | Male | 1.45 (1.01-2.08), | 1.00 (0.69-1.47), |
| | | p = 0.05 | p = 0.98 |
| | Female | 1.00 | 1.00 |
| Margins | Negative | 1.00 | 1.00 |
| | Positive | 1.83 (1.16-2.90), | 1.56 (0.98-2.49), |
| | | p = 0.01 | p = 0.06 |
| Stage | Stage 1 | 1.00 | |
| | Stage 2 | 4.40 (1.49-12.99), | 4.39 (1.48-12.97), |
| | | p = 0.01 | p = 0.01 |
| | Stage 3 | 11.99 (4.35-33.04), | 12.29 (4.45-33.98), |
| | | p < 0.001 | p < 0.001 |
| | Stage 4 | 30.13 (10.78-84.22), | 28.56 (10.14-80.43), |
| | | p < 0.001 | p < 0.001 |

TABLE 3
Interaction between the G-INT and G-DIF subtypes and benefit from 5-FU
based adjuvant treatment. Cox proportional hazards regression for survival was used
to evaluate interactions between the intrinsic subtypes and 5-FU adjuvant treatment, in
patients eligible for adjuvant 5-FU based therapy. Hazard ratios are adjusted for stage.

| | | | HR (95% CI), p | |
|---|---|---|---|---|
| | G-INT | G-DIF | value | p value for |
| | (deaths/N) | (deaths/N) | (G-INT: HR = 1.0) | interaction |
| Adjuvant 5-FU | 20/45 (44%) | 29/38 (76%) | 2.71 (1.52-4.85), | P = 0.002 |
| based-treatment | | | p = 0.001 | |
| Surgery alone | 49/136 (36%) | 48/86 (56%) | 1.37 (0.92-2.05), | |
| | | | p = 0.12 | |
| HR (95% CI), | 1.68 (0.98-2.88), | 0.90 (0.56-1.45), | | |
| p value | p = 0.06 | p = 0.67 | | |
| (5-FU based | | | | |
| therapy, HR = 1) | | | | |

TABLE 4

| | Genomic | Genomic | HR (95% CI), p value | p value for |
|---|---|---|---|---|
| | Subtype 1 | Subtype 2 | (Subset 1: HR = 1.0) | interaction |
| Adjuvant 5-FU | | | Model 1 | H₀: |
| based-treatment | | | exp(a_{g=2; t=1})/exp(a_{g=1; t=1}) | c_{g=1;t=1} = |
| Surgery alone | | | Model 2 | c_{g=1;t=2} = |
| | | | exp(a_{g=2; t=2})/exp(a_{g=1; t=2}) | c_{g=2;t=1} = |
| HR (95% CI), | Model 3 | Model 4 | | c_{g=2;t=2} = 0 |
| p value | exp(b_{t=2; g=1})/ | exp(b_{t=2; g=2})/ | | H_{A}: At least 1 |
| (5-FU based | exp(b_{t=1; g=1}) | exp(b_{t=1; g=2}) | | interaction |
| therapy, HR = 1) | | | | term is not |
| | | | | zero (c_{g=i;t=j} ≠ 0) |

TABLE 5
LIMMA identifies 171 genes distinguishing G-INT and G-DIF subtypes.

| | | |
|---|---|---|
| | | Adjusted p |
| Gene Symbol | Gene Title | value |

Genes upregulated in G-INT

| | | |
|---|---|---|
| TSPAN8 | tetraspanin 8 | 7.38E−09 |
| GPX2 | glutathione peroxidase 2 (gastrointestinal) | 1.00E−07 |
| LYZ | lysozyme (renal amyloidosis) | 2.40E−07 |
| PLS1 | plastin 1 (I isoform) | 1.18E−06 |
| LGALS4 | lectin | 1.18E−06 |
| FUT2 | fucosyltransferase 2 (secretor status included) | 5.01E−06 |
| C5orf32 | chromosome 5 open reading frame 32 | 5.01E−06 |
| ATAD4 | ATPase family | 1.08E−05 |
| DEGS2 | degenerative spermatocyte homolog 2 | 1.08E−05 |
| NOSTRIN | nitric oxide synthase trafficker | 1.20E−05 |
| MUC13 | mucin 13 | 2.71E−05 |
| ALDH3A1 | aldehyde dehydrogenase 3 family | 2.84E−05 |
| MYO1A | myosin IA | 3.58E−05 |
| ABCC3 | ATP-binding cassette | 4.12E−05 |
| AGR3 | anterior gradient homolog 3 (Xenopus laevis) | 5.69E−05 |
| VILL | villin-like | 5.69E−05 |
| SH3RF1 | SH3 domain containing ring finger 1 | 7.53E−05 |
| TRAK1 | trafficking protein | 8.57E−05 |
| EGLN3 | egl nine homolog 3 (C. elegans) | 9.49E−05 |
| CDH17 | cadherin 17 | 0.0001 |
| BCL2L14 | BCL2-like 14 (apoptosis facilitator) | 0.0001 |
| CEACAM1 | carcinoembryonic antigen-related cell adhesion | 0.0001 |
| | molecule 1 (biliary glycoprotein) | |
| LIPH | lipase | 0.0001 |
| RSPH1 | radial spoke head 1 homolog (Chlamydomonas) | 0.0001 |
| KALRN | kalirin | 0.0002 |
| CAPN8 | calpain 8 | 0.0002 |
| CLCN3 | Chloride channel 3 | 0.0002 |
| PLEK2 | pleckstrin 2 | 0.0002 |
| TMC5 | transmembrane channel-like 5 | 0.0002 |
| CYP3A5 | cytochrome P450 | 0.0002 |
| EPS8L3 | EPS8-like 3 | 0.0002 |
| FA2H | fatty acid 2-hydroxylase | 0.0002 |
| TOX3 | TOX high mobility group box family member 3 | 0.0002 |
| BAIAP2L2 | BAI1-associated protein 2-like 2 | 0.0003 |
| PIP5K1B | phosphatidylinositol-4-phosphate 5-kinase | 0.0003 |
| AGPAT2 | 1-acylglycerol-3-phosphate O-acyltransferase 2 | 0.0003 |
| | (lysophosphatidic acid acyltransferase | |
| BCL2L15 | BCL2-like 15 | 0.0003 |
| TNFRSF11A | tumor necrosis factor receptor superfamily | 0.0003 |
| PLCH1 | phospholipase C | 0.0004 |
| GPR35 | G protein-coupled receptor 35 | 0.0004 |
| ATP10B | ATPase | 0.0004 |
| TC2N | tandem C2 domains | 0.0004 |
| MMP28 | matrix metallopeptidase 28 | 0.0004 |
| CYP3A5 | cytochrome P450 | 0.0005 |
| LLGL2 | lethal giant larvae homolog 2 (Drosophila) | 0.0005 |
| CAPN10 | calpain 10 | 0.0005 |
| TRNP1 | TMF1-regulated nuclear protein 1 | 0.0005 |
| SDCBP2 | syndecan binding protein (syntenin) 2 | 0.0006 |
| MYB | v-myb myeloblastosis viral oncogene homolog | 0.0006 |
| | (avian) | |
| ACSM3 | acyl-CoA synthetase medium-chain family member 3 | 0.0006 |
| REG4 | regenerating islet-derived family | 0.0007 |
| CYP2C18 | cytochrome P450 | 0.0008 |
| PRR15 | proline rich 15 | 0.0008 |
| SGK493 | protein kinase-like protein SgK493 | 0.0009 |
| HNF4G | hepatocyte nuclear factor 4 | 0.0009 |
| TMEM45B | transmembrane protein 45B | 0.0009 |
| KLF5 | Kruppel-like factor 5 (intestinal) | 0.0009 |
| UGT8 | UDP glycosyltransferase 8 | 0.0009 |
| RNF128 | ring finger protein 128 | 0.0009 |
| KCNE3 | potassium voltage-gated channel | 0.0009 |
| LOC100133019 | similar to hCG-int983765 | 0.0009 |
| DNAJC22 | DnaJ (Hsp40) homolog | 0.0009 |
| ST6GALNAC1 | ST6 (alpha-N-acetyl-neuraminyl-2 | 0.0009 |
| CLRN3 | clarin 3 | 0.0010 |
| GDF15 | growth differentiation factor 15 | 0.0010 |
| RNF43 | ring finger protein 43 | 0.0010 |
| KIAA0746 | KIAA0746 protein | 0.0011 |
| USH1C | Usher syndrome 1C (autosomal recessive | 0.0011 |
| CLDN2 | claudin 2 | 0.0013 |
| EHF | Ets homologous factor | 0.0013 |
| FOXA3 | forkhead box A3 | 0.0014 |
| POF1B | premature ovarian failure | 0.0014 |
| LOC286208 | hypothetical LOC286208 | 0.0014 |
| C9orf152 | chromosome 9 open reading frame 152 | 0.0015 |
| GMDS | GDP-mannose 4 | 0.0015 |
| SLC22A18AS | solute carrier family 22 (organic cation transporter) | 0.0016 |
| C11orf9 | chromosome 11 open reading frame 9 | 0.0016 |
| LOC100131701 | hypothetical protein LOC100131701 | 0.0016 |
| TMPRSS4 | transmembrane protease | 0.0016 |
| SLC37A1 | solute carrier family 37 (glycerol-3-phosphate | 0.0016 |
| | transporter) | |
| PTK6 | PTK6 protein tyrosine kinase 6 | 0.0016 |
| CEACAM5 | carcinoembryonic antigen-related cell adhesion | 0.0017 |
| | molecule 5 | |
| SULT2B1 | sulfotransferase family | 0.0017 |
| LOC120376 | Uncharacterized protein LOC120376 | 0.0018 |
| MST1R | macrophage stimulating 1 receptor (c-met-related | 0.0018 |
| | tyrosine kinase) | |
| ELF3 | E74-like factor 3 (ets domain transcription factor | 0.0018 |
| SLC26A9 | solute carrier family 26 | 0.0019 |
| SLC40A1 | solute carrier family 40 (iron-regulated transporter) | 0.0019 |
| PTPRB | protein tyrosine phosphatase | 0.0019 |
| AGR2 | anterior gradient homolog 2 (Xenopus laevis) | 0.0019 |
| GALNT12 | UDP-N-acetyl-alpha-D-galactosamine:polypeptide | 0.0019 |
| | N-acetylgalactosaminyltransferase 12 (GalNAc- | |
| | T12) | |
| HEPH | hephaestin | 0.0019 |

Genes upregulated in G-DIF

| | | |
|---|---|---|
| RDX | radixin | 2.26E−09 |
| TBCEL | Tubulin folding cofactor E-like | 3.58E−08 |
| FERMT2 | fermitin family homolog 2 (Drosophila) | 7.47E−08 |
| MYO5A | myosin VA (heavy chain 12 | 4.25E−07 |
| SOAT1 | sterol O-acyltransferase 1 | 1.08E−06 |
| FADS1 | fatty acid desaturase 1 | 7.87E−06 |
| MYH10 | myosin | 1.05E−05 |
| FNBP1 | formin binding protein 1 | 1.15E−05 |
| ELOVL5 | ELOVL family member 5 | 1.43E−05 |
| ABL2 | v-abl Abelson murine leukemia viral oncogene | 3.99E−05 |
| | homolog 2 (arg | |
| PGBD1 | piggyBac transposable element derived 1 | 6.09E−05 |
| SELM | selenoprotein M | 8.84E−05 |
| LOXL2 | lysyl oxidase-like 2 | 0.0001 |
| c(“N-PAC” | “SEPT6”) | 0.0001 |
| FZD2 | frizzled homolog 2 (Drosophila) | 0.0002 |
| KIAA1586 | KIAA1586 | 0.0002 |
| RASSF8 | Ras association (RalGDS/AF-6) domain family (N- | 0.0002 |
| | terminal) member 8 | |
| NUAK1 | NUAK family | 0.0002 |
| TMEFF1 | transmembrane protein with EGF-like and two | 0.0002 |
| | follistatin-like domains 1 | |
| SCHIP1 | schwannomin interacting protein 1 | 0.0002 |
| TMEM136 | transmembrane protein 136 | 0.0002 |
| ZCCHC11 | zinc finger | 0.0002 |
| FAM101B | family with sequence similarity 101 | 0.0002 |
| FAM127A | family with sequence similarity 127 | 0.0002 |
| SIX4 | SIX homeobox 4 | 0.0003 |
| DENND5A | DENN/MADD domain containing 5A | 0.0003 |
| TTC7B | tetratricopeptide repeat domain 7B | 0.0003 |
| ZNF512B | zinc finger protein 512B | 0.0003 |
| KIRREL | kin of IRRE like (Drosophila) | 0.0003 |
| GNB4 | guanine nucleotide binding protein (G protein) | 0.0003 |
| FN1 | fibronectin 1 | 0.0004 |
| GJC1 | gap junction protein | 0.0004 |
| GLIPR2 | GLI pathogenesis-related 2 | 0.0005 |
| FJX1 | four jointed box 1 (Drosophila) | 0.0006 |
| DSE | dermatan sulfate epimerase | 0.0006 |
| ENAH | enabled homolog (Drosophila) | 0.0007 |
| DNAH14 | dynein | 0.0007 |
| CALD1 | caldesmon 1 | 0.0008 |
| GPRASP2 | G protein-coupled receptor associated sorting protein 2 | 0.0008 |
| HEG-int | HEG homolog 1 (zebrafish) | 0.0009 |
| DLX1 | distal-less homeobox 1 | 0.0009 |
| TIMP3 | TIMP metallopeptidase inhibitor 3 | 0.0009 |
| GLT8D4 | glycosyltransferase 8 domain containing 4 | 0.0009 |
| LPHN2 | latrophilin 2 | 0.0009 |
| PTPRS | Protein tyrosine phosphatase | 0.0009 |
| FRMD6 | FERM domain containing 6 | 0.0009 |
| SNAP47 | synaptosomal-associated protein | 0.0009 |
| c(“WHAMML1” | “WHAMML2”) | 0.0010 |
| GATA2 | GATA binding protein 2 | 0.0010 |
| APH1B | anterior pharynx defective 1 homolog B (C. elegans) | 0.0010 |
| MLLT11 | myeloid/lymphoid or mixed-lineage leukemia (trithorax | 0.0010 |
| | homolog | |
| PPM1F | protein phosphatase 1F (PP2C domain containing) | 0.0013 |
| SNX21 | sorting nexin family member 21 | 0.0013 |
| ANXA6 | annexin A6 | 0.0014 |
| PKIG | protein kinase (cAMP-dependent | 0.0014 |
| ANTXR1 | anthrax toxin receptor 1 | 0.0015 |
| ATP8B2 | ATPase | 0.0015 |
| CSRP2 | cysteine and glycine-rich protein 2 | 0.0015 |
| DEGS1 | degenerative spermatocyte homolog 1 | 0.0017 |
| KLHDC8B | kelch domain containing 8B | 0.0017 |
| DEPDC1 | DEP domain containing 1 | 0.0018 |
| CSE1L | CSE1 chromosome segregation 1-like (yeast) | 0.0018 |
| WDR35 | WD repeat domain 35 | 0.0018 |
| SAMD4A | sterile alpha motif domain containing 4A | 0.0018 |
| TRIM23 | tripartite motif-containing 23 | 0.0018 |
| FAM92A1 | family with sequence similarity 92 | 0.0018 |
| S1PR3 | sphingosine-1-phosphate receptor 3 | 0.0018 |
| TUBA1A | tubulin | 0.0018 |
| LOC644450 | hypothetical protein LOC644450 | 0.0018 |
| PTPN1 | protein tyrosine phosphatase | 0.0018 |
| HOMER3 | homer homolog 3 (Drosophila) | 0.0018 |
| IGFBP7 | insulin-like growth factor binding protein 7 | 0.0018 |
| TSR1 | TSR1 | 0.0018 |
| AURKB | aurora kinase B | 0.0019 |
| MSX1 | msh homeobox 1 | 0.0019 |
| CTSL1 | cathepsin L1 | 0.0019 |
| TEAD1 | TEA domain family member 1 (SV40 transcriptional | 0.0019 |
| | enhancer factor) | |
| LOC283658 | hypothetical protein LOC283658 | 0.0020 |
| MAP1B | microtubule-associated protein 1B | 0.0020 |

TABLE 6
Gene ontology biological processes enriched among genes upregulated
in G-INT/G-DIF subtypes.

| | | |
|---|---|---|
| | Fisher | |
| Gene ontology Biological | Exact | Within-system |
| Process | probability | FDR |

G-INT

| | | |
|---|---|---|
| carbohydrate metabolism | 0.03 | 0.00 |
| protein biosynthesis | 0.03 | 0.00 |
| macromolecule biosynthesis | 0.05 | 0.00 |
| protein amino acid glycosylation | 0.07 | 0.07 |
| cell-cell adhesion | 0.07 | 0.06 |
| glycoprotein metabolism | 0.07 | 0.06 |
| electron transport | 0.07 | 0.05 |
| glycoprotein biosynthesis | 0.07 | 0.05 |

G-DIF

| | | |
|---|---|---|
| fatty acid metabolism | 0.02 | 0.00 |
| intracellular transport | 0.02 | 0.00 |
| cell growth | 0.02 | 0.00 |
| cell proliferation | 0.03 | 0.00 |
| protein transport | 0.07 | 0.04 |
| protein targeting | 0.07 | 0.04 |
| fatty acid desaturation | 0.07 | 0.04 |
| cell growth and/or maintenance | 0.07 | 0.03 |
| response to | | |
| pest/pathogen/parasite | 0.07 | 0.05 |
| intracellular protein transport | 0.07 | 0.05 |

TABLE 7
Clinical Characteristics of Patient Cohorts and Correlation to G-INT and G-DIF Subtypes.
Correlation of G-INT and G-DIF primary tumors to clinical, demographic and pathologic variables in the
four cohorts. p value for age was determined by a t-test, all other p values are determined by chi-square
tests. Median follow-up for patients still alive for the 4 cohorts are 33, 56, 39 and 36 months respectively.

| | | | | | | | | | | | | All 4 |
|---|---|---|---|---|---|---|---|---|---|---|---|---|
| SG | | | AU | | | YG | | | TMA | | | cohorts |
| G-INT | G-DIF | P- | G-INT | G-DIF | P- | G-INT | G-DIF | P- | G-INT | G-DIF | P | P- |
| (N = 113) | (N = 84) | value | (N = 38) | (N = 32) | value | (N = 35) | (N = 30) | value | (N = 75) | (N = 44) | value | value |

Age

| | | | | | | | | | | | | | |
|---|---|---|---|---|---|---|---|---|---|---|---|---|---|
| range | 23-92 | 27-83 | 0.53 | 32-85 | 33-85 | 0.34 | 34-83 | 32-80 | 0.96 | 33-87 | 31-87 | 0.1 | 0.62 |
| mean, S.D | 65.8, 13.5 | 63.9, 12.6 | | 66.9, 12.5 | 64.0, 12.6 | | 61.0, 11.9 | 60.9, 11.2 | | 64.4, 12.1 | 68.2, 12.1 | | |

Gender

| | | | | | | | | | | | | | |
|---|---|---|---|---|---|---|---|---|---|---|---|---|---|
| Male | 75 | 53 | 0.63 | 26 | 22 | 0.98 | 22 | 24 | 0.13 | 51 | 29 | 0.84 | 0.88 |
| Female | 38 | 31 | | 12 | 10 | | 13 | 6 | | 24 | 15 | | |

Lauren's

| | | | | | | | | | | | | | |
|---|---|---|---|---|---|---|---|---|---|---|---|---|---|
| Intestinal | 69 | 31 | 0.002 | 22 | 12 | 0.003 | 11 | 11 | 0.26 | 34 | 27 | 0.09 | <0.001 |
| Diffuse | 32 | 44 | | 10 | 20 | | 15 | 16 | | 20 | 12 | | |
| Mixed | 12 | 9 | | 6 | 0 | | 9 | 3 | | 21 | 5 | | |

Grade

| | | | | | | | | | | | | | |
|---|---|---|---|---|---|---|---|---|---|---|---|---|---|
| Moderate | 48 | 24 | 0.05 | 18 | 6 | 0.01 | 20 | 20 | 0.59 | 24 | 12 | 0.59 | 0.04 |
| to well | | | | | | | | | | | | | |
| differentiated | | | | | | | | | | | | | |
| Poorly | 65 | 60 | | 20 | 26 | | 15 | 10 | | 51 | 32 | | |
| differentiated | | | | | | | | | | | | | |

Stage

| | | | | | | | | | | | | | |
|---|---|---|---|---|---|---|---|---|---|---|---|---|---|
| 1 | 20 | 11 | 0.36 | 9 | 4 | 0.53 | 8 | 4 | 0.11 | 7 | 1 | 0.15 | 0.12* |
| 2 | 20 | 12 | | 8 | 8 | | 2 | 0 | | 22 | 21 | | |
| 3 | 43 | 29 | | 18 | 15 | | 20 | 15 | | 25 | 13 | | |
| 4 | 30 | 32 | | 3 | 5 | | 5 | 11 | | 21 | 9 | | |

Adjuvant 5-FU based therapy (in eligible patients)***

| | | | | | | | | | | | |
|---|---|---|---|---|---|---|---|---|---|---|---|
| Yes | 19 | 17 | 0.33 | 15 | 13 | 0.27 | Not available | 11 | 8 | 0.96 | 0.27** |
| No | 76 | 47 | | 21 | 10 | | Not available | 39 | 29 | | |

Surgical Margins

| | | | | | | | | | | | |
|---|---|---|---|---|---|---|---|---|---|---|---|
| Negative | 99 | 70 | 0.40 | 37 | 29 | 0.23 | Not available | 65 | 41 | 0.37 | 0.66 |
| Positive | 14 | 14 | | 1 | 3 | | Not available | 10 | 3 | | |

*chi-square test when stage groups are combined, stage 1-2 vs stage 3-4: p = 0.3, stage 1, 2, 3 vs stage 4: p = 0.08
**chi-square test for each stage: stage 1: 0.81, stage 2: p = 0.74, stage 3: p = 0.64, stage 4 p = 0.43
***Stage distribution among patients receiving 5FU (stage 1: 3, Stage 2: 19, Stage 3: 43, Stage 4: 18); Stage distribution among patients treated with surgery alone (Stage 1: 30, Stage 2: 65, Stage 3: 93, Stage 4: 34); chi-square test, p = 0.03

TABLE 8
Interaction between G-INT/G-DIF status and benefit from 5-
FU based adjuvant treatment. Cox proportional hazards regression for survival
was used to evaluate interactions between the intrinsic subtypes as determined
by Gene expression (Cohort 1 & 2) and by Tissue microarray (Cohort 4) and
5-FU adjuvant treatment, in patients eligible for adjuvant 5-Fluorouracil based
therapy. Hazard ratios are adjusted for stage.

| | | | HR (95% CI), p | |
|---|---|---|---|---|
| | G-INT | G-DIF | value | p value for |
| | (deaths/N) | (deaths/N) | (G-INT: HR = 1.0) | interaction |
| Gene expression: | | | | |
| Cohort 1 & 2 | | | | |
| Adjuvant 5-FU | 17/34 (50%) | 24/30 (80%) | 2.30 (1.22-4.32), | p = 0.03 |
| based-treatment | | | p = 0.01 | |
| Surgery alone | 35/97 (36%) | 31/57 (54%) | 1.28 (0.78-2.09), | |
| | | | p = 0.33 | |
| HR (95% CI), | 1.52 (0.82-2.79), | 0.86 (0.50-1.49), | | |
| p value | p = 0.18 | p = 0.59 | | |
| (5-FU based | | | | |
| therapy, HR = 1) | | | | |
| Tissue | | | | |
| microarray: | | | | |
| Cohort 4 | | | | |
| Adjuvant 5-FU | 3/11 (27%) | 5/8 (63%) | 5.04 (1.07-23.7), | p = 0.02 |
| based-treatment | | | p = 0.04 | |
| Surgery alone | 14/39 (36%) | 17/29 (58%) | 1.49 (0.72-3.09), | |
| | | | p = 0.29 | |
| HR (95% CI), | 2.82 (0.80-10.00), | 0.96 (0.35-2.65), | | |
| p value | p = 0.11 | p = 0.95 | | |
| (5-FU based | | | | |
| therapy, HR = 1) | | | | |

TABLE 9
Bioinformatics Data 1

| # | ID | | logFC | AveExpr | t | P. Value | adj. P. Val | B |
|---|---|---|---|---|---|---|---|---|
| 1 | 204969_s_{—} | RDX | −3.12748 | 7.649716 | −10.8734 | 2.23E−13 | 2.26E−09 | 19.84673 |
| 2 | 203824_at | TSPAN8 | 6.409965 | 9.796255 | 10.19428 | 1.46E−12 | 7.38E−09 | 18.13375 |
| 3 | 227395_at | TBCEL | −2.81073 | 6.535276 | −9.49847 | 1.06E−11 | 3.58E−08 | 16.3066 |
| 4 | 209210_s_{—} | FERMT2 | −4.86275 | 8.040461 | −9.14861 | 2.95E−11 | 7.47E−08 | 15.36085 |
| 5 | 202831_at | GPX2 | 5.414887 | 9.478959 | 8.973513 | 4.95E−11 | 1.00E−07 | 14.88092 |
| 6 | 213975_s_{—} | LYZ | 5.799997 | 7.625607 | 8.620725 | 1.42E−10 | 2.40E−07 | 13.90088 |
| 7 | 227761_at | MYO5A | −2.73065 | 6.818824 | −8.37996 | 2.94E−10 | 4.25E−07 | 13.22235 |
| 8 | 221561_at | SOAT1 | −3.37041 | 7.4237 | −8.03008 | 8.54E−10 | 1.08E−06 | 12.22296 |
| 9 | 205190_at | PLS1 | 2.367 | 10.36055 | 7.938261 | 1.13E−09 | 1.18E−06 | 11.95818 |
| 10 | 204272_at | LGALS4 | 5.024427 | 8.247304 | 7.93033 | 1.16E−09 | 1.18E−06 | 11.93526 |
| 11 | 210608_s_{—} | FUT2 | 2.126299 | 8.190536 | 7.411169 | 5.83E−09 | 5.01E−06 | 10.4198 |
| 12 | 224707_at | C5orf32 | 1.746987 | 10.9226 | 7.405748 | 5.93E−09 | 5.01E−06 | 10.40383 |
| 13 | 208962_s_{—} | FADS1 | −3.0292 | 7.864197 | −7.23641 | 1.01E−08 | 7.87E−06 | 9.903421 |
| 14 | 212372_at | MYH10 | −3.75029 | 8.831142 | −7.11983 | 1.46E−08 | 1.05E−05 | 9.557426 |
| 15 | 219127_at | ATAD4 | 2.976132 | 7.906676 | 7.083657 | 1.63E−08 | 1.08E−05 | 9.44982 |
| 16 | 236496_at | DEGS2 | 1.411009 | 7.086113 | 7.069496 | 1.71E−08 | 1.08E−05 | 9.407665 |
| 17 | 212288_at | FNBP1 | −2.31476 | 8.061822 | −7.03063 | 1.93E−08 | 1.15E−05 | 9.291877 |
| 18 | 226992_at | NOSTRIN | 2.508938 | 6.57373 | 7.00035 | 2.12E−08 | 1.20E−05 | 9.201605 |
| 19 | 208788_at | ELOVL5 | −4.98683 | 8.773705 | −6.92656 | 2.68E−08 | 1.43E−05 | 8.981283 |
| 20 | 218687_s_{—} | MUC13 | 2.888096 | 8.104833 | 6.709857 | 5.34E−08 | 2.71E−05 | 8.332058 |
| 21 | 205623_at | ALDH3A1 | 3.634132 | 8.744173 | 6.679033 | 5.89E−08 | 2.84E−05 | 8.239465 |
| 22 | 211916_s_{—} | MYO1A | 1.415163 | 6.564923 | 6.591652 | 7.78E−08 | 3.58E−05 | 7.976676 |
| 23 | 231907_at | ABL2 | −1.35748 | 8.128956 | −6.54393 | 9.06E−08 | 3.99E−05 | 7.832977 |
| 24 | 208161_s_{—} | ABCC3 | 2.926107 | 9.425609 | 6.520662 | 9.75E−08 | 4.12E−05 | 7.762884 |
| 25 | 228241_at | AGR3 | 4.706808 | 6.496131 | 6.402726 | 1.42E−07 | 5.69E−05 | 7.407184 |
| 26 | 209950_s_{—} | VILL | 2.039712 | 7.373369 | 6.394592 | 1.46E−07 | 5.69E−05 | 7.38263 |
| 27 | 235411_at | PGBD1 | −1.41284 | 5.242617 | −6.36136 | 1.62E−07 | 6.09E−05 | 7.282285 |
| 28 | 225589_at | SH3RF1 | 1.743039 | 8.124842 | 6.283315 | 2.08E−07 | 7.53E−05 | 7.046481 |
| 29 | 201283_s_{—} | TRAK1 | 1.501586 | 6.714547 | 6.232072 | 2.45E−07 | 8.57E−05 | 6.891554 |
| 30 | 226051_at | SELM | −2.33842 | 8.070815 | −6.2117 | 2.62E−07 | 8.84E−05 | 6.829941 |
| 31 | 219232_s_{—} | EGLN3 | 2.232631 | 6.856834 | 6.179386 | 2.90E−07 | 9.49E−05 | 6.73219 |
| 32 | 209847_at | CDH17 | 4.073017 | 8.176292 | 6.063821 | 4.20E−07 | 0.000133 | 6.382444 |
| 33 | 221241_s_{—} | BCL2L14 | 1.70139 | 6.648793 | 6.055302 | 4.32E−07 | 0.000133 | 6.356655 |
| 34 | 209498_at | CEACAM1 | 3.331116 | 8.687292 | 6.040843 | 4.52E−07 | 0.000133 | 6.312879 |
| 35 | 202998_s_{—} | LOXL2 | −3.12066 | 6.921788 | −6.03535 | 4.60E−07 | 0.000133 | 6.296249 |
| 36 | 235871_at | LIPH | 1.939163 | 7.653389 | 6.023976 | 4.77E−07 | 0.000134 | 6.261815 |
| 37 | 230093_at | RSPH1 | 1.648657 | 6.494428 | 6.011421 | 4.97E−07 | 0.000136 | 6.2238 |
| 38 | 212414_s_{—} | 38961 | −2.09632 | 7.34951 | −5.97959 | 5.50E−07 | 0.000147 | 6.127425 |
| 39 | 210220_at | FZD2 | −2.22561 | 8.362122 | −5.9572 | 5.91E−07 | 0.000152 | 6.059625 |
| 40 | 227750_at | KALRN | 1.729873 | 8.505005 | 5.952671 | 6.00E−07 | 0.000152 | 6.045911 |
| 41 | 231869_at | KIAA1586 | −1.57723 | 6.441973 | −5.9109 | 6.85E−07 | 0.000169 | 5.91943 |
| 42 | 229030_at | CAPN8 | 1.915576 | 5.806245 | 5.894662 | 7.22E−07 | 0.000174 | 5.870262 |
| 43 | 201734_at | CLCN3 | 1.417702 | 9.890141 | 5.881904 | 7.52E−07 | 0.000177 | 5.831633 |
| 44 | 218644_at | PLEK2 | 1.890949 | 9.6378 | 5.86588 | 7.92E−07 | 0.000182 | 5.783115 |
| 45 | 240304_s_{—} | TMC5 | 3.9222 | 8.508619 | 5.850297 | 8.32E−07 | 0.000187 | 5.735932 |
| 46 | 225946_at | RASSF8 | −2.88883 | 6.48773 | −5.83627 | 8.70E−07 | 0.000192 | 5.693473 |
| 47 | 204589_at | NUAK1 | −2.18879 | 7.459694 | −5.79373 | 9.97E−07 | 0.000213 | 5.564682 |
| 48 | 205122_at | TMEFF1 | −2.44367 | 6.648816 | −5.78947 | 1.01E−06 | 0.000213 | 5.551791 |
| 49 | 205765_at | CYP3A5 | 3.160494 | 6.859158 | 5.76699 | 1.09E−06 | 0.000222 | 5.483747 |
| 50 | 204030_s_{—} | SCHIP1 | −2.7224 | 7.581643 | −5.76473 | 1.09E−06 | 0.000222 | 5.476897 |
| 51 | 1554076_s_{—} | TMEM136 | −1.03491 | 7.157822 | −5.74395 | 1.17E−06 | 0.000229 | 5.414034 |
| 52 | 212704_at | ZCCHC11 | −1.28881 | 7.96818 | −5.73673 | 1.20E−06 | 0.000229 | 5.392168 |
| 53 | 226905_at | FAM101B | −3.67556 | 6.910626 | −5.73618 | 1.20E−06 | 0.000229 | 5.390492 |
| 54 | 219404_at | EPS8L3 | 2.547745 | 7.166897 | 5.723793 | 1.25E−06 | 0.000234 | 5.353024 |
| 55 | 201828_x_{—} | FAM127A | −2.07478 | 9.84709 | −5.7129 | 1.29E−06 | 0.000238 | 5.320056 |
| 56 | 219429_at | FA2H | 2.765245 | 7.736621 | 5.703687 | 1.33E−06 | 0.000239 | 5.292193 |
| 57 | 216623_x_{—} | TOX3 | 3.949084 | 6.419557 | 5.700587 | 1.34E−06 | 0.000239 | 5.282815 |
| 58 | 229796_at | SIX4 | −1.6395 | 7.643161 | −5.67818 | 1.44E−06 | 0.000252 | 5.215031 |
| 59 | 212561_at | DENND5A | −2.11688 | 9.111193 | −5.66637 | 1.50E−06 | 0.000257 | 5.179313 |
| 60 | 221178_at | BAIAP2L2 | 1.672783 | 5.559754 | 5.645955 | 1.60E−06 | 0.00027 | 5.117574 |
| 61 | 226152_at | TTC7B | −2.1599 | 7.063461 | −5.63011 | 1.68E−06 | 0.000278 | 5.069661 |
| 62 | 55872_at | ZNF512B | −2.46183 | 8.139271 | −5.6273 | 1.70E−06 | 0.000278 | 5.061168 |
| 63 | 225303_at | KIRREL | −2.10247 | 6.381472 | −5.6062 | 1.82E−06 | 0.000292 | 4.997373 |
| 64 | 225710_at | GNB4 | −4.16344 | 6.314512 | −5.60028 | 1.85E−06 | 0.000293 | 4.979502 |
| 65 | 205632_s_{—} | PIP5K1B | 3.37937 | 7.693746 | 5.595648 | 1.88E−06 | 0.000293 | 4.965497 |
| 66 | 32837_at | AGPAT2 | 1.127766 | 9.982 | 5.5715 | 2.03E−06 | 0.000312 | 4.892527 |
| 67 | 242013_at | BCL2L15 | 2.145616 | 4.895326 | 5.56191 | 2.09E−06 | 0.000317 | 4.863552 |
| 68 | 238846_at | TNFRSF11A | 2.932551 | 6.528316 | 5.53377 | 2.29E−06 | 0.000341 | 4.77856 |
| 69 | 211719_x_{—} | FN1 | −4.62486 | 8.842298 | −5.51309 | 2.45E−06 | 0.000359 | 4.716123 |
| 70 | 214745_at | PLCH1 | 1.669389 | 6.085065 | 5.497569 | 2.57E−06 | 0.000372 | 4.669267 |
| 71 | 210264_at | GPR35 | 1.691186 | 8.014079 | 5.482601 | 2.70E−06 | 0.000385 | 4.624095 |
| 72 | 228776_at | GJC1 | −3.07741 | 6.982209 | −5.47429 | 2.77E−06 | 0.00039 | 4.599024 |
| 73 | 214070_s_{—} | ATP10B | 2.400287 | 7.44816 | 5.466078 | 2.84E−06 | 0.000394 | 4.57424 |
| 74 | 1553132_a | TC2N | 2.928399 | 7.093906 | 5.437718 | 3.11E−06 | 0.000426 | 4.488708 |
| 75 | 239272_at | MMP28 | 2.09676 | 5.979179 | 5.417812 | 3.31E−06 | 0.000448 | 4.428695 |
| 76 | 225604_s_{—} | GLIPR2 | −1.51972 | 5.907997 | −5.39453 | 3.57E−06 | 0.000476 | 4.358522 |
| 77 | 214234_s_{—} | CYP3A5 | 2.902548 | 7.387218 | 5.380782 | 3.73E−06 | 0.000491 | 4.317116 |
| 78 | 203713_s_{—} | LLGL2 | 1.378389 | 7.933217 | 5.360681 | 3.98E−06 | 0.000517 | 4.256582 |
| 79 | 221040_at | CAPN10 | 1.528718 | 4.377891 | 5.341642 | 4.22E−06 | 0.000537 | 4.199269 |
| 80 | 227862_at | TRNP1 | 2.030153 | 8.8084 | 5.340431 | 4.24E−06 | 0.000537 | 4.195625 |
| 81 | 219522_at | FJX1 | −1.9392 | 7.691573 | −5.32109 | 4.51E−06 | 0.000556 | 4.137424 |
| 82 | 218854_at | DSE | −3.31209 | 7.194195 | −5.32073 | 4.52E−06 | 0.000556 | 4.136351 |
| 83 | 233565_s_{—} | SDCBP2 | 1.739595 | 8.923597 | 5.318043 | 4.55E−06 | 0.000556 | 4.128262 |
| 84 | 204798_at | MYB | 1.761055 | 7.213209 | 5.287921 | 5.01E−06 | 0.000605 | 4.037684 |
| 85 | 210377_at | ACSM3 | 2.440852 | 6.543656 | 5.264235 | 5.40E−06 | 0.000644 | 3.966502 |
| 86 | 217820_s_{—} | ENAH | −1.63772 | 9.02446 | −5.2528 | 5.60E−06 | 0.000655 | 3.932153 |
| 87 | 242283_at | DNAH14 | −2.38614 | 6.756808 | −5.25166 | 5.62E−06 | 0.000655 | 3.928742 |
| 88 | 1554436_a | REG4 | 2.925995 | 5.832288 | 5.231551 | 6.00E−06 | 0.000691 | 3.868348 |
| 89 | 208126_s_{—} | CYP2C18 | 2.326414 | 6.115446 | 5.19621 | 6.71E−06 | 0.000764 | 3.762308 |
| 90 | 212077_at | CALD1 | −4.17479 | 8.590961 | −5.17204 | 7.24E−06 | 0.000812 | 3.689861 |
| 91 | 228027_at | GPRASP2 | −1.62283 | 7.14916 | −5.16985 | 7.29E−06 | 0.000812 | 3.683286 |
| 92 | 226961_at | PRR15 | 2.267782 | 7.40636 | 5.155546 | 7.63E−06 | 0.000841 | 3.640426 |
| 93 | 225380_at | SGK493 | 1.694835 | 8.55337 | 5.144174 | 7.91E−06 | 0.000859 | 3.606367 |
| 94 | 213069_at | HEG1 | −2.6251 | 8.290619 | −5.13769 | 8.08E−06 | 0.000859 | 3.586948 |
| 95 | 242138_at | DLX1 | −2.1522 | 5.444525 | −5.13015 | 8.27E−06 | 0.000859 | 3.564382 |
| 96 | 201150_s_{—} | TIMP3 | −3.64291 | 6.270025 | −5.12844 | 8.32E−06 | 0.000859 | 3.559251 |
| 97 | 232271_at | HNF4G | 2.204372 | 5.986147 | 5.126023 | 8.38E−06 | 0.000859 | 3.552028 |
| 98 | 230323_s_{—} | TMEM45B | 3.240195 | 8.24453 | 5.120909 | 8.52E−06 | 0.000859 | 3.536722 |
| 99 | 235371_at | GLT8D4 | −2.26511 | 6.821543 | −5.12002 | 8.54E−06 | 0.000859 | 3.534077 |
| 100 | 209212_s_{—} | KLF5 | 2.402545 | 9.954668 | 5.118503 | 8.58E−06 | 0.000859 | 3.529522 |
| 101 | 206953_s_{—} | LPHN2 | −3.2915 | 5.997597 | −5.11756 | 8.61E−06 | 0.000859 | 3.52671 |
| 102 | 229465_s_{—} | PTPRS | −1.95511 | 7.772765 | −5.11613 | 8.65E−06 | 0.000859 | 3.522427 |
| 103 | 228956_at | UGT8 | 2.983756 | 7.168536 | 5.112976 | 8.73E−06 | 0.000859 | 3.512987 |
| 104 | 219263_at | RNF128 | 4.373142 | 8.77983 | 5.108166 | 8.87E−06 | 0.000864 | 3.498597 |
| 105 | 227647_at | KCNE3 | 2.929789 | 7.498027 | 5.09944 | 9.12E−06 | 0.000875 | 3.4725 |
| 106 | 225464_at | FRMD6 | −3.12681 | 7.941138 | −5.09829 | 9.15E−06 | 0.000875 | 3.469074 |
| 107 | 1559125_a | LOC100133 | 1.029492 | 3.972168 | 5.092231 | 9.33E−06 | 0.000883 | 3.450944 |
| 108 | 220441_at | DNAJC22 | 1.661796 | 7.534604 | 5.080019 | 9.69E−06 | 0.000908 | 3.414443 |
| 109 | 225244_at | SNAP47 | −0.69953 | 9.265949 | −5.07581 | 9.82E−06 | 0.000908 | 3.401856 |
| 110 | 227725_at | ST6GALNAC | 3.076038 | 6.366759 | 5.074969 | 9.85E−06 | 0.000908 | 3.399351 |
| 111 | 229777_at | CLRN3 | 3.620969 | 7.082285 | 5.053569 | 1.05E−05 | 0.000956 | 3.335429 |
| 112 | 221577_x_{—} | GDF15 | 3.343072 | 9.404597 | 5.052998 | 1.06E−05 | 0.000956 | 3.333724 |
| 113 | 1557261_a | WHAMML2 | −0.94243 | 4.798474 | −5.03771 | 1.11E−05 | 0.000994 | 3.288077 |
| 114 | 209710_at | GATA2 | −1.56731 | 8.031497 | −5.02812 | 1.14E−05 | 0.001007 | 3.259479 |
| 115 | 218704_at | RNF43 | 2.035613 | 8.271949 | 5.028026 | 1.14E−05 | 0.001007 | 3.259194 |
| 116 | 221036_s_{—} | APH1B | −0.9404 | 7.189307 | −5.01127 | 1.20E−05 | 0.001047 | 3.209231 |
| 117 | 211071_s_{—} | MLLT11 | −2.58229 | 8.14195 | −5.01031 | 1.21E−05 | 0.001047 | 3.20636 |
| 118 | 212314_at | KIAA0746 | 2.715466 | 9.174234 | 4.98923 | 1.29E−05 | 0.001109 | 3.143536 |
| 119 | 211184_s_{—} | USH1C | 2.21404 | 7.213818 | 4.983899 | 1.31E−05 | 0.001119 | 3.127655 |
| 120 | 223509_at | CLDN2 | 2.185491 | 6.39057 | 4.941373 | 1.50E−05 | 0.001264 | 3.001095 |
| 121 | 203063_at | PPM1F | −0.83208 | 7.327591 | −4.93984 | 1.51E−05 | 0.001264 | 2.996546 |
| 122 | 225645_at | EHF | 4.251009 | 9.065455 | 4.926573 | 1.57E−05 | 0.001307 | 2.957099 |
| 123 | 1553960_a | SNX21 | −1.79264 | 6.621707 | −4.92096 | 1.60E−05 | 0.00132 | 2.940431 |
| 124 | 200982_s_{—} | ANXA6 | −1.85341 | 7.613982 | −4.90808 | 1.67E−05 | 0.001353 | 2.902163 |
| 125 | 228463_at | FOXA3 | 2.14789 | 7.237209 | 4.908009 | 1.67E−05 | 0.001353 | 2.901948 |
| 126 | 1555383_a | POF1B | 2.636332 | 6.416991 | 4.900356 | 1.71E−05 | 0.001375 | 2.879227 |
| 127 | 202732_at | PKIG | −2.07537 | 7.993264 | −4.89781 | 1.72E−05 | 0.001375 | 2.871665 |
| 128 | 1560089_a | LOC286208 | 1.297152 | 7.616368 | 4.889413 | 1.77E−05 | 0.001401 | 2.846748 |
| 129 | 224694_at | ANTXR1 | −3.14361 | 5.953627 | −4.87283 | 1.86E−05 | 0.001459 | 2.797563 |
| 130 | 229964_at | C9orf152 | 2.70188 | 5.687052 | 4.869654 | 1.88E−05 | 0.001459 | 2.788142 |
| 131 | 204875_s_{—} | GMDS | 2.256813 | 9.171042 | 4.869131 | 1.89E−05 | 0.001459 | 2.78659 |
| 132 | 226771_at | ATP8B2 | −2.32395 | 6.010242 | −4.8574 | 1.96E−05 | 0.001502 | 2.751815 |
| 133 | 207030_s_{—} | CSRP2 | −2.27802 | 7.717543 | −4.84889 | 2.01E−05 | 0.001531 | 2.726594 |
| 134 | 206097_at | SLC22A18A | 0.783331 | 8.208614 | 4.839348 | 2.07E−05 | 0.001559 | 2.698347 |
| 135 | 204073_s_{—} | C11orf9 | 1.803489 | 8.202639 | 4.837345 | 2.08E−05 | 0.001559 | 2.692417 |
| 136 | 238804_at | LOC100131 | 1.218681 | 5.819783 | 4.836176 | 2.09E−05 | 0.001559 | 2.688957 |
| 137 | 218960_at | TMPRSS4 | 2.36773 | 8.496208 | 4.81931 | 2.21E−05 | 0.001631 | 2.639045 |
| 138 | 218928_s_{—} | SLC37A1 | 1.151477 | 7.698334 | 4.814165 | 2.24E−05 | 0.001638 | 2.623824 |
| 139 | 206482_at | PTK6 | 2.141604 | 7.220746 | 4.813342 | 2.25E−05 | 0.001638 | 2.621391 |
| 140 | 209250_at | DEGS1 | −1.37671 | 9.713911 | −4.80582 | 2.30E−05 | 0.001665 | 2.599147 |
| 141 | 225755_at | KLHDC8B | −1.24311 | 6.462643 | −4.7888 | 2.43E−05 | 0.001737 | 2.548849 |
| 142 | 201884_at | CEACAM5 | 3.74779 | 8.106504 | 4.78737 | 2.44E−05 | 0.001737 | 2.544628 |
| 143 | 205759_s_{—} | SULT2B1 | 1.465931 | 6.487127 | 4.7857 | 2.45E−05 | 0.001737 | 2.539696 |
| 144 | 220295_x_{—} | DEPDC1 | −1.29119 | 8.224061 | −4.77854 | 2.51E−05 | 0.001764 | 2.518538 |
| 145 | 201111_at | CSE1L | −1.2016 | 10.74747 | −4.77561 | 2.53E−05 | 0.001768 | 2.509897 |
| 146 | 226890_at | WDR35 | −1.02158 | 6.098493 | −4.77044 | 2.57E−05 | 0.001783 | 2.494636 |
| 147 | 228338_at | LOC120376 | 2.096359 | 6.459132 | 4.768402 | 2.59E−05 | 0.001783 | 2.488624 |
| 148 | 205455_at | MST1R | 1.413022 | 8.043291 | 4.766042 | 2.61E−05 | 0.001784 | 2.48166 |
| 149 | 210827_s_{—} | ELF3 | 2.084691 | 9.710752 | 4.758857 | 2.67E−05 | 0.001813 | 2.460464 |
| 150 | 212845_at | SAMD4A | −1.52973 | 8.420734 | −4.75328 | 2.71E−05 | 0.001826 | 2.444021 |
| 151 | 204732_s_{—} | TRIM23 | −0.97146 | 6.526184 | −4.74919 | 2.75E−05 | 0.001826 | 2.43196 |
| 152 | 235391_at | FAM92A1 | −2.66439 | 7.488703 | −4.74824 | 2.76E−05 | 0.001826 | 2.429162 |
| 153 | 228176_at | S1PR3 | −2.07828 | 5.657533 | −4.7433 | 2.80E−05 | 0.001826 | 2.414605 |
| 154 | 209118_s_{—} | TUBA1A | −3.52897 | 8.165291 | −4.74194 | 2.81E−05 | 0.001826 | 2.410576 |
| 155 | 222347_at | LOC644450 | −0.86798 | 6.086172 | −4.73823 | 2.84E−05 | 0.001826 | 2.39965 |
| 156 | 202716_at | PTPN1 | −0.95332 | 8.531469 | −4.73784 | 2.85E−05 | 0.001826 | 2.398509 |
| 157 | 204647_at | HOMER3 | −0.98943 | 7.293145 | −4.73597 | 2.86E−05 | 0.001826 | 2.392984 |
| 158 | 201163_s_{—} | IGFBP7 | −4.08287 | 6.343352 | −4.73577 | 2.86E−05 | 0.001826 | 2.392393 |
| 159 | 221987_s_{—} | TSR1 | −0.90261 | 7.957029 | −4.73573 | 2.86E−05 | 0.001826 | 2.392291 |
| 160 | 242271_at | SLC26A9 | 1.629691 | 6.396131 | 4.722526 | 2.99E−05 | 0.00187 | 2.353391 |
| 161 | 223044_at | SLC40A1 | 3.401386 | 8.520135 | 4.72252 | 2.99E−05 | 0.00187 | 2.353372 |
| 162 | 209464_at | AURKB | −0.95327 | 8.727687 | −4.72039 | 3.01E−05 | 0.00187 | 2.347091 |
| 163 | 230250_at | PTPRB | 1.846357 | 5.553639 | 4.71865 | 3.02E−05 | 0.00187 | 2.341978 |
| 164 | 205932_s_{—} | MSX1 | −1.55938 | 7.455975 | −4.71794 | 3.03E−05 | 0.00187 | 2.339892 |
| 165 | 209173_at | AGR2 | 4.449875 | 10.34674 | 4.715091 | 3.06E−05 | 0.00187 | 2.331502 |
| 166 | 218885_s_{—} | GALNT12 | 2.146879 | 8.591959 | 4.71432 | 3.06E−05 | 0.00187 | 2.329233 |
| 167 | 202087_s_{—} | CTSL1 | −1.73528 | 9.730728 | −4.7092 | 3.11E−05 | 0.001889 | 2.314162 |
| 168 | 224955_at | TEAD1 | −1.09304 | 10.47158 | −4.70371 | 3.17E−05 | 0.00191 | 2.298027 |
| 169 | 203903_s_{—} | HEPH | 3.188783 | 5.779323 | 4.695347 | 3.25E−05 | 0.001949 | 2.27342 |
| 170 | 239741_at | LOC283658 | −1.07882 | 4.133079 | −4.68692 | 3.34E−05 | 0.001981 | 2.248635 |
| 171 | 226084_at | MAP1B | −3.11821 | 6.047552 | −4.68639 | 3.34E−05 | 0.001981 | 2.247 |

TABLE 10
Bioinformatics Data 2

| No. of | Total | Accuracy | | Precision | | |
|---|---|---|---|---|---|---|
| Criteria | Matches | (out of 59) | (out of 55) | p < 00.5 | p < 00.1 | Notes |
| 171 | 70 | 59 | 55 | 59 | 55 | |
| 170 | 70 | 59 | 55 | 58 | 56 | |
| 169 | 70 | 59 | 55 | 58 | 56 | |
| 168 | 70 | 59 | 55 | 58 | 56 | |
| 167 | 70 | 59 | 55 | 58 | 56 | |
| 166 | 70 | 59 | 55 | 58 | 56 | |
| 165 | 70 | 59 | 55 | 58 | 55 | |
| 164 | 70 | 59 | 55 | 59 | 55 | |
| 163 | 70 | 59 | 55 | 58 | 55 | |
| 162 | 70 | 59 | 55 | 59 | 54 | |
| 161 | 70 | 59 | 55 | 59 | 54 | |
| 160 | 70 | 59 | 55 | 58 | 53 | |
| 159 | 70 | 59 | 55 | 59 | 55 | |
| 158 | 70 | 59 | 55 | 59 | 55 | |
| 157 | 70 | 59 | 55 | 60 | 55 | |
| 156 | 70 | 59 | 55 | 59 | 55 | |
| 155 | 70 | 59 | 55 | 59 | 54 | |
| 154 | 70 | 59 | 55 | 59 | 54 | |
| 153 | 70 | 59 | 55 | 59 | 54 | |
| 152 | 70 | 59 | 55 | 59 | 54 | |
| 151 | 70 | 59 | 55 | 59 | 55 | |
| 150 | 70 | 59 | 55 | 57 | 51 | |
| 149 | 70 | 59 | 55 | 58 | 55 | |
| 148 | 70 | 59 | 55 | 58 | 54 | |
| 147 | 70 | 59 | 55 | 58 | 52 | |
| 146 | 70 | 59 | 55 | 58 | 55 | |
| 145 | 70 | 59 | 55 | 59 | 55 | |
| 144 | 70 | 59 | 55 | 59 | 55 | |
| 143 | 70 | 59 | 55 | 59 | 55 | |
| 142 | 69 | 59 | 55 | 59 | 54 | a |
| 141 | 69 | 59 | 55 | 59 | 54 | |
| 140 | 69 | 59 | 55 | 59 | 53 | |
| 139 | 69 | 59 | 55 | 59 | 55 | |
| 138 | 69 | 59 | 55 | 60 | 54 | |
| 137 | 69 | 59 | 55 | 59 | 54 | |
| 136 | 69 | 59 | 55 | 59 | 54 | |
| 135 | 69 | 59 | 55 | 60 | 54 | |
| 134 | 69 | 59 | 55 | 60 | 54 | |
| 133 | 69 | 59 | 55 | 60 | 55 | |
| 132 | 69 | 59 | 55 | 60 | 54 | |
| 131 | 68 | 59 | 55 | 59 | 53 | |
| 130 | 69 | 59 | 55 | 59 | 53 | |
| 129 | 69 | 59 | 55 | 60 | 53 | |
| 128 | 69 | 59 | 55 | 59 | 52 | |
| 127 | 68 | 59 | 55 | 59 | 53 | a |
| 126 | 68 | 59 | 55 | 59 | 54 | |
| 125 | 68 | 59 | 55 | 53 | 44 | |
| 124 | 68 | 59 | 55 | 59 | 52 | |
| 123 | 68 | 59 | 55 | 59 | 53 | |
| 122 | 68 | 59 | 55 | 59 | 52 | |
| 121 | 68 | 59 | 55 | 59 | 53 | |
| 120 | 68 | 59 | 55 | 58 | 51 | |
| 119 | 68 | 59 | 55 | 58 | 53 | |
| 118 | 68 | 59 | 55 | 58 | 54 | |
| 117 | 68 | 59 | 55 | 59 | 52 | |
| 116 | 68 | 59 | 55 | 58 | 51 | |
| 115 | 68 | 59 | 55 | 59 | 52 | |
| 114 | 68 | 59 | 55 | 59 | 53 | |
| 113 | 68 | 59 | 55 | 59 | 53 | |
| 112 | 68 | 59 | 55 | 59 | 52 | |
| 111 | 68 | 59 | 55 | 58 | 53 | |
| 110 | 68 | 59 | 55 | 58 | 52 | |
| 109 | 68 | 59 | 55 | 58 | 53 | |
| 108 | 68 | 59 | 55 | 58 | 53 | |
| 107 | 68 | 59 | 55 | 59 | 53 | |
| 106 | 68 | 59 | 55 | 58 | 54 | |
| 105 | 68 | 59 | 55 | 58 | 53 | |
| 104 | 68 | 59 | 55 | 58 | 53 | |
| 103 | 68 | 59 | 55 | 58 | 53 | |
| 102 | 68 | 59 | 55 | 58 | 53 | |
| 101 | 68 | 59 | 55 | 58 | 54 | |
| 100 | 68 | 59 | 55 | 55 | 41 | |
| 99 | 68 | 59 | 55 | 58 | 54 | |
| 98 | 67 | 59 | 55 | 58 | 52 | a |
| 97 | 67 | 59 | 55 | 58 | 53 | |
| 96 | 67 | 59 | 55 | 58 | 53 | |
| 95 | 67 | 59 | 55 | 58 | 51 | |
| 94 | 67 | 59 | 55 | 58 | 52 | |
| 93 | 67 | 59 | 55 | 58 | 52 | |
| 92 | 67 | 59 | 55 | 58 | 51 | |
| 91 | 67 | 59 | 55 | 59 | 52 | |
| 90 | 67 | 59 | 55 | 58 | 51 | |
| 89 | 67 | 59 | 55 | 60 | 50 | |
| 88 | 67 | 59 | 55 | 58 | 50 | |
| 87 | 67 | 59 | 55 | 58 | 51 | |
| 86 | 67 | 59 | 55 | 59 | 50 | |
| 85 | 67 | 59 | 55 | 57 | 50 | |
| 84 | 67 | 59 | 55 | 57 | 50 | |
| 83 | 67 | 59 | 55 | 59 | 50 | |
| 82 | 67 | 59 | 55 | 57 | 50 | |
| 81 | 67 | 59 | 55 | 58 | 49 | |
| 80 | 67 | 59 | 55 | 55 | 40 | |
| 79 | 67 | 59 | 55 | 57 | 50 | |
| 78 | 67 | 59 | 55 | 57 | 50 | |
| 77 | 67 | 59 | 55 | 56 | 50 | |
| 76 | 67 | 59 | 55 | 56 | 50 | |
| 75 | 67 | 59 | 55 | 56 | 45 | |
| 74 | 67 | 59 | 55 | 56 | 50 | |
| 73 | 67 | 59 | 55 | 54 | 50 | |
| 72 | 67 | 59 | 55 | 56 | 50 | |
| 71 | 67 | 59 | 55 | 58 | 51 | |
| 70 | 67 | 59 | 55 | 55 | 49 | |
| 69 | 67 | 59 | 55 | 59 | 50 | |
| 68 | 67 | 59 | 55 | 56 | 48 | |
| 67 | 67 | 59 | 55 | 56 | 49 | |
| 66 | 68 | 59 | 55 | 57 | 47 | |
| 65 | 68 | 59 | 55 | 56 | 47 | |
| 64 | 67 | 59 | 55 | 55 | 45 | |
| 63 | 67 | 59 | 55 | 55 | 46 | |
| 62 | 68 | 59 | 55 | 56 | 46 | |
| 61 | 68 | 59 | 55 | 56 | 44 | |
| 60 | 68 | 59 | 55 | 53 | 42 | |
| 59 | 68 | 59 | 55 | 57 | 45 | |
| 58 | 68 | 59 | 55 | 56 | 43 | |
| 57 | 68 | 59 | 55 | 56 | 43 | |
| 56 | 68 | 59 | 55 | 53 | 43 | |
| 55 | 68 | 59 | 55 | 55 | 43 | |
| 54 | 68 | 59 | 55 | 55 | 43 | |
| 53 | 68 | 59 | 55 | 56 | 43 | |
| 52 | 68 | 59 | 55 | 54 | 39 | |
| 51 | 68 | 59 | 55 | 54 | 40 | |
| 50 | 68 | 59 | 55 | 47 | 31 | |
| 49 | 68 | 59 | 55 | 54 | 40 | |
| 48 | 68 | 59 | 55 | 53 | 36 | |
| 47 | 68 | 59 | 55 | 55 | 39 | |
| 46 | 67 | 58 | 55 | 52 | 37 | b |
| 45 | 67 | 58 | 55 | 52 | 35 | |
| 44 | 67 | 58 | 55 | 51 | 36 | |
| 43 | 67 | 58 | 55 | 49 | 37 | |
| 42 | 67 | 58 | 55 | 48 | 37 | |
| 41 | 67 | 58 | 55 | 48 | 37 | |
| 40 | 67 | 58 | 55 | 41 | 29 | |
| 39 | 68 | 59 | 55 | 50 | 35 | |
| 38 | 67 | 58 | 55 | 45 | 36 | |
| 37 | 67 | 58 | 55 | 46 | 35 | |
| 36 | 67 | 58 | 55 | 41 | 35 | |
| 35 | 67 | 58 | 55 | 43 | 33 | |
| 34 | 67 | 58 | 55 | 44 | 34 | |
| 33 | 67 | 58 | 55 | 43 | 36 | |
| 32 | 67 | 58 | 55 | 43 | 28 | |
| 31 | 67 | 58 | 55 | 44 | 36 | |
| 30 | 67 | 58 | 55 | 46 | 29 | |
| 29 | 67 | 58 | 55 | 47 | 36 | |
| 28 | 67 | 58 | 55 | 44 | 29 | |
| 27 | 68 | 59 | 55 | 47 | 30 | |
| 26 | 66 | 58 | 55 | 47 | 28 | |
| 25 | 67 | 59 | 55 | 42 | 21 | |
| 24 | 67 | 59 | 55 | 46 | 25 | |
| 23 | 67 | 59 | 55 | 45 | 30 | |
| 22 | 67 | 59 | 55 | 43 | 27 | |
| 21 | 67 | 59 | 55 | 42 | 22 | |
| 20 | 68 | 59 | 55 | 32 | 7 | c |
| 19 | 67 | 59 | 55 | 36 | 22 | |
| 18 | 67 | 59 | 55 | 35 | 18 | |
| 17 | 67 | 59 | 55 | 30 | 15 | |
| 16 | 67 | 58 | 55 | 29 | 7 | |
| 15 | 66 | 58 | 55 | 28 | 9 | a |
| 14 | 66 | 58 | 55 | 27 | 8 | |
| 13 | 66 | 58 | 55 | 23 | 0 | |
| 12 | 65 | 57 | 55 | 17 | 0 | a |
| 11 | 65 | 57 | 55 | 16 | 0 | |
| 10 | 66 | 58 | 55 | 0 | 0 | |
| 9 | 64 | 57 | 54 | 2 | 0 | a |
| 8 | 65 | 57 | 54 | 0 | 0 | |
| 7 | 65 | 58 | 55 | 1 | 0 | |
| 6 | 63 | 57 | 55 | 0 | 0 | a |
| 5 | 63 | 56 | 53 | 0 | 0 | b |
| 4 | Error | Error | Error | Error | Error | |
| 3 | Error | Error | Error | Error | Error | |
| 2 | Error | Error | Error | Error | Error | |
| 1 | Error | Error | Error | Error | Error | |

Notes:
a Drop in accuracy (out of original 70)
b Drop in accuracy (out of original 59)
c Drop in precision (significant change)

TABLE 11
Intrinsic Signature Applied to 549 Primary Tumors in 6 Independent
Datasets

| | | | Percentage |
|---|---|---|---|
| Patient Cohort and | | Total Classified by | Classified |
| Microarray | Sample Size | NTP at FDR <0.05 | Confidently |
| Singapore | | | |
| Affymetrix U133- | 197 | 174 | 88.3 |
| 2plus microarray | | | |
| Australia | | | |
| Affymetrix U133- | 70 | 62 | 88.6 |
| 2 plus microarray | | | |
| Hong Kong | | | |
| Custom microarray | 90 | 55 | 61.1 |
| United Kingdom | | | |
| Affymetrix U133AB | 31 | 24 | 77.4 |
| microarray | | | |
| Korea set 1 | | | |
| Custom microarray | 96 | 69 | 71.9 |
| Korea set 2 | | | |
| Illumina Human-6 | 65 | 48 | 73.8 |
| v2 microarray | | | |
| Total | 549 | 432 | 78.6 |

The nearest template prediction algorithm was used to map the 171 gene set onto 6 microarray datasets comprising 549 primary tumors profiled on different platforms. 78.6% of the tumors were classified precisely at a false discovery rate of 5%. In contrast, with 5 other published signatures, classification precision was <30%.

TABLE 12
Comparisons of the Intrinsic Subtypes Classification with Lauren's
Histology and Stage

| Factor | HR | p-value |
|---|---|---|
| Intrinsic Subtypes | 1.49 | 0.01 |
| Lauren's histology | 1.11 | 0.49 |
| Stage | 1.99 | <0.01 |

1. A method of diagnosing intestinal-type gastric cancer (G-INT) or diffuse-type gastric cancer (G-DIF), the method comprising the step of:
determining the expression levels of the following Group A1 genes in gastric tissue in a biological sample from a subject having gastric cancer: TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2 and TMC5, and optionally determining the expression level of at least one of the following Group A2 genes in the biological sample: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 and HEPH,
wherein an increase in the expression levels of the Group A1 and optional Group A2 genes in the subject, compared to expression levels of the genes in non-cancerous gastric tissue, indicates that the subject has G-INT;
or
determining the expression levels of the following Group B1 genes in gastric tissue in a biological sample from a subject having gastric cancer: RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586 and RASSF8, and optionally determining the expression level of at least one of the following Group B2 genes in the biological sample: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 and MAP1B,
wherein an increase in the expression levels of the Group B1 and optional Group B2 genes in the subject, compared to expression levels of the genes in non-cancerous gastric tissue, indicates that the subject has G-DIF.

2. The method of claim 1, wherein the expression level of at least one of the following additional genes is also determined: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 or HEPH.

3. The method of claim 2, wherein the expression levels of at least ten of the additional genes are also determined.

4. The method of claim 1, wherein the expression level of at least one of the following additional genes is also determined: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 or MAP1B.

5. The method of claim 4, wherein the expression levels of at least ten of the additional genes are also determined.

6. The method of claim 1, wherein the biological sample is a gastric tissue biopsy obtained endoscopically.

7. A method for prognosis of gastric cancer in a subject, the method comprising the steps of:
(a) determining the expression levels of the following Group A1 genes in gastric tissue in a biological sample from a subject having gastric cancer: TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2 and TMC5, and optionally determining the expression level of at least one of the following Group A2 genes in the biological sample: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, Cllorf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 and HEPH; and
(b) determining the expression levels of the following Group B1 genes in gastric tissue in a biological sample from a subject having gastric cancer: RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586 and RASSF8, and optionally determining the expression level of at least one of the following Group B2 genes in the biological sample: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 and MAP1B;
wherein an increase in the expression levels of the Group A1 and optional Group A2 genes in the subject, compared to expression levels of the genes in non-cancerous gastric tissue, indicates that the subject has G-INT, and wherein an increase in the expression levels of the Group B1 and optional Group B2 genes in the subject, compared to expression levels of the genes in non-cancerous gastric tissue, indicates that the subject has G-DIF.

8. The method of claim 7, wherein the expression level of at least one of the following additional genes is also determined: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 or HEPH.

9. The method of claim 8, wherein the expression levels of at least ten of the additional genes are also determined.

10. The method of claim 7, wherein the expression level of at least one of the following additional genes is also determined: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 or MAP1B.

11. The method of claim 10, wherein the expression levels of at least ten of the additional genes are also determined.

12. The method of claim 7, wherein the biological sample is a gastric tissue biopsy obtained endoscopically.

13. A method of treating gastric cancer in a subject, the method comprising the steps of:
(a) determining whether the subject has intestinal-type gastric cancer (G-INT) or diffuse-type gastric cancer (G-DIF) according to the method of claim 1; and
(b) administering a chemotherapeutic agent to the subject.

14. A method of treating gastric cancer in a subject, the method comprising the steps of:
(a) determining whether the subject has intestinal-type gastric cancer (G-INT) or diffuse-type gastric cancer (G-DIF) according to the method of claim 1; and
(b) if the subject has G-INT as determined in step (a), administering 5-fluorouracil or an oral fluoropyrimidine, and/or oxaliplatin to the subject;
(c) if the subject has G-DIF as determined in step (a), administering cisplatin to the subject.

15. An array comprising a set of polynucleotide probes, wherein the set of polynucleotide probes are:
specific for the expression products of the following Group A1 genes: TSPAN8, GPX2, LYZ, PLS1, LGALS4, FUT2, C5orf32, ATAD4, DEGS2, NOSTRIN, MUC13, ALDH3A1, MYO1A, ABCC3, AGR3, VILL, SH3RF1, TRAK1, EGLN3, CDH17, BCL2L14, CEACAM1, LIPH, RSPH1, KALRN, CAPN8, CLCN3, PLEK2 and TMC5, and optionally the expression product of at least one of the following Group A2 genes: CYP3A5, EPS8L3, FA2H, TOX3, BAIAP2L2, PIP5K1B, AGPAT2, BCL2L15, TNFRSF11A, PLCH1, GPR35, ATP10B, TC2N, MMP28, CYP3A5, LLGL2, CAPN10, TRNP1, SDCBP2, MYB, ACSM3, REG4, CYP2C18, PRR15, SGK493, HNF4G, TMEM45B, KLF5, UGT8, RNF128, KCNE3, LOC100133019, DNAJC22, ST6GALNAC1, CLRN3, GDF15, RNF43, KIAA0746, USH1C, CLDN2, EHF, FOXA3, POF1B, LOC286208, C9orf152, GMDS, SLC22A18AS, C11orf9, LOC100131701, TMPRSS4, SLC37A1, PTK6, CEACAM5, SULT2B1, LOC120376, MST1R, ELF3, SLC26A9, SLC40A1, PTPRB, AGR2, GALNT12 and HEPH; and/or
specific for the expression products of the following Group B1 genes: RDX, TBCEL, FERMT2, MYO5A, SOAT1, FADS1, MYH10, FNBP1, ELOVL5, ABL2, PGBD1, SELM, LOXL2, cN-PAC, FZD2, KIAA1586 and RASSF8, and optionally the expression product of at least one of the following Group B2 genes: NUAK1, TMEFF1, SCHIP1, TMEM136, ZCCHC11, FAM101B, FAM127A, SIX4, DENND5A, TTC7B, ZNF512B, KIRREL, GNB4, FN1, GJC1, GLIPR2, FJX1, DSE, ENAH, DNAH14, CALD1, GPRASP2, HEG-int, DLX1, TIMP3, GLT8D4, LPHN2, PTPRS, FRMD6, SNAP47, WHAMML1, WHAMML2, GATA2, APH1B, MLLT11, PPM1F, SNX21, ANXA6, PKIG, ANTXR1, ATP8B2, CSRP2, DEGS1, KLHDC8B, DEPDC1, CSE1L, WDR35, SAMD4A, TRIM23, FAM92A1, S1PR3, TUBA1A, LOC644450, PTPN1, HOMER3, IGFBP7, TSR1, AURKB, MSX1, CTSL1, TEAD1, LOC283658 and MAP1B;
and wherein the set of polynucleotide probes do not include probes specific for expression products of genes other than the Groups A1, A2, B1 and B2 genes.

16. The array of claim 15, wherein the set of polynucleotide probes further comprises probes that are specific for the expression products of at least one additional Group A2 genes.

17. The array of claim 16, wherein the set of polynucleotide probes further comprises probes that are specific for the expression products of at least ten of the additional Group A2 genes.

18. The array of claim 15, wherein the set of polynucleotide probes further comprises probes that are able specific for the expression products of at least one additional Group B2 genes.

19. The array of claim 18, wherein the set of polynucleotide probes further comprises probes that are able specific for the expression products of at least ten of the additional Group B2 genes.

20. The array of claim 15, wherein the set of polynucleotides are specific for the expression products of the Group A1 genes and the Group B1 genes.
